(19) United States
(12) Patent Application Publication
(10) Pub. No.: US 2023/0287067 A1
(43) Pub. Date: Sep. 14, 2023

(54) HUMAN IMMUNOGENIC EPITOPES OF HEMO AND HHLA2 HUMAN ENDOGENOUS RETROVIRUSES (HERVS)

(71) Applicant: The United States of America,as represented by the Secretary,Department of Health and Human Services, Bethesda, MD (US)

(72) Inventors: Jeffrey Schlom, Potomac, MD (US); Duane H. Hamilton, Washington, DC (US); Claudia M. Palena, Potomac, MD (US); Renee N. Donahue, Germantown, MD (US)

(73) Assignee: The United States of America,as represented by the Secretary,Department of Health and Human Services, Bethesda, MD (US)

(21) Appl. No.: 17/793,753

(22) PCT Filed: Jan. 21, 2021
(86) PCT No.: PCT/US2021/014335

§ 371 Date: Jul. 19, 2022

Related U.S. Application Data
(60) Provisional application No. 62/963,872, filed on Jan. 21, 2020.

(51) Int. Cl. C07K 14/47; C12N 15/85; A61K 45/06; A61K 9/127; A61K 35/17; A61P 35/00

(52) U.S. Cl. CPC C07K 14/47; C12N 15/85; A61K 45/06; A61K 9/127; A61K 35/17; A61P 35/00

(57) ABSTRACT

The invention provides human immunogenic epitopes of HEMO and HHLA2 human endogenous retroviruses (HERVs), which can be used as a peptide, polypeptide (protein), and/or in a vaccine or other composition for the prevention or therapy of cancer. The invention further provides a nucleic acid encoding the peptide or polypeptide (protein), a vector comprising the nucleic acid, a cell comprising the peptide, polypeptide (protein), nucleic acid, or vector, and compositions thereof.

### CROSS-REFERENCE TO RELATED APPLICATIONS

This patent application is a United States national phase of International Patent Application PCT/US2021/014335, filed on Jan. 21, 2021. This patent application claims the benefit of U.S. Provisional Patent Application No. 62/963,872, filed on Jan. 21, 2020, which is incorporated by reference.

### STATEMENT REGARDING

### Federally Sponsored Research or Development

This invention was made with Government support under project number ZIABC010944 by the National Institutes of Health, National Cancer Institute. The Government has certain rights in the invention.

### SEQUENCE LISTING

Incorporated by reference in its entirety herein is a computer-readable nucleotide/amino acid sequence listing submitted concurrently herewith and identified as follows: One 23,933 Byte ASCII (Text) file named “763522_ST25.TXT,” created on Jul. 12, 2022.

### BACKGROUND OF THE INVENTION

Human Endogenous Retroviruses (HERVs) are remnant of retrovirus germ line infections early in primate evolution and are not viruses. Remarkably, HERVs represent approximately 8% of the human genome. They are an extremely diverse group constituted by at least 3 major classes: Class I consisting of HERV-H, HHLA2 and HERV-E, among others; Class II consisting of HERV-K, among others; and a very diverse Class III. HERV-ERVMER34-1 (HEMO) has not yet been classified. HERVs encode env and gag sequences with a similar organization to Retroviridae, and they can be epigenetically regulated.

There is a desire to identify human immunogenic epitopes HEMO AND HHLA2 HERVS for treatment of cancer patients.

### BRIEF SUMMARY OF THE INVENTION

The invention provides a peptide comprising, consisting essentially of, or consisting of the amino acid sequence of any one of SEQ ID NOs: 1-100 and 103. In particular, the invention provides a peptide comprising, consisting essentially of, or consisting of the amino acid sequence of any one of SEQ ID NOs: 7-12, 20-28, 34-39, 53-59, 77-85, and 96-100.

In another aspect, the invention provides a polypeptide (protein) comprising the peptide; a nucleic acid encoding the peptide; a vector comprising the nucleic acid; a cell comprising the peptide, polypeptide (protein), nucleic acid, or vector; and compositions thereof.

The invention provides a method of inhibiting cancer in a subject comprising administering a therapeutically effective amount of a composition comprising the peptide, polypeptide (protein), nucleic acid, vector, or cell to the subject to the subject, wherein cancer in the subject is inhibited.

The invention also provides a method of enhancing an immune response against cancer in a subject comprising administering a therapeutically effective amount of a composition comprising the peptide, polypeptide (protein), nucleic acid, vector, or cell to the subject, wherein the immune response in the subject is enhanced.

The invention also provides a method of treating cancer in a subject comprising administering a therapeutically effective amount of a composition comprising the peptide, polypeptide (protein), nucleic acid, vector, or cell to the subject.

The invention also provides a method of reducing, arresting, reversing or preventing the metastatic progression of cancer in a subject comprising administering a therapeutically effective amount of a composition comprising the peptide, polypeptide (protein), nucleic acid, vector, or cell to the subject.

The invention also provides a method of preventing or delaying the onset of cancer in a subject comprising administering a therapeutically effective amount of a composition comprising the peptide, polypeptide (protein), nucleic acid, vector, or cell to the subject.

The invention further provides a method of inhibiting cancer in a subject comprising (a) obtaining (isolating) lymphocytes from the subject, (b) stimulating the lymphocytes with a composition comprising the peptide, polypeptide (protein), nucleic acid, vector, or cell to the subject to generate cytotoxic T lymphocytes ex vivo, and (c) administering the cytotoxic T lymphocytes to the subject, wherein cancer in the subject is inhibited.

The invention provides a method of inhibiting cancer in a subject comprising (a) obtaining (isolating) dendritic cells from the subject, (b) treating the dendritic cells with a composition comprising the peptide, polypeptide (protein), nucleic acid, vector, or cell ex vivo, and (c) administering the treated dendritic cells to the subject, wherein cancer in the subject is inhibited.

Additionally, the invention provides inhibiting cancer in a subject comprising (a) obtaining peripheral blood mononuclear cells (PBMCs) from a subject suffering from cancer, (b) isolating dendritic cells from the PBMCs, (c) treating the dendritic cells with a composition comprising the peptide, polypeptide (protein), nucleic acid, vector, or cell ex vivo, (d) activating the PBMCs with the treated dendritic cells ex vivo, and (e) administering the activated PBMCs to the subject, wherein cancer in the subject is inhibited.

The invention further provides inhibiting cancer in a subject comprising (a) obtaining peripheral blood mononuclear cells (PBMCs) from a subject suffering from cancer, (b) isolating dendritic cells from the PBMCs, (c) treating the dendritic cells with a composition comprising the peptide, polypeptide (protein), nucleic acid, vector, or cell ex vivo, (d) activating the PBMCs with the treated dendritic cells ex vivo, (e) isolating T lymphocytes from the activated PBMCs ex vivo, and (f) administering the isolated T lymphocytes to the subject, wherein cancer in the subject is inhibited.

The invention provides the use of adoptively transferred T cells stimulated in vitro with a composition comprising the peptide, polypeptide (protein), nucleic acid, vector, or cell to treat cancer, to inhibit cancer, to reduce, arrest, reverse, or prevent the metastatic progression of cancer in a subject, or to prevent or delay the onset of cancer.

In an additional aspect, the invention provides a method of inducing an immune response against cancer in a subject comprising (a) administering to the subject a first vector (e.g., viral vector, such as poxviral vector) comprising a nucleic acid encoding the amino acid sequence of any one of SEQ ID NOs: 1-100 and 103 and (b) administering to the subject a second vector (e.g., viral vector, such as poxviral vector) comprising a nucleic acid encoding the amino acid sequence of any one of SEQ ID NOs: 1-100 and 103.

The invention also provides a method for inhibiting cancer in a subject comprising administering T cell receptor (TCR) engineered T cells or TCR engineered NK cells to the subject, wherein the TCR recognizes one or more epitopes of HEMO and/or HHLA2 human endogenous retroviruses (HERVs).

### BRIEF DESCRIPTION OF THE SEVERAL VIEWS OF THE DRAWINGS

FIGS. 1A-1F demonstrate the expression of HEMO and HHLA in normal and cancer tissues.

FIG. 1A depicts the RNA expression (transcripts per million) of indicated transcripts in normal human tissues (GTExPortal database hosted by the Broad Institute). In particular, increased RNA expression of HHLA2 was observed in colon and small intestine, as well as testis and kidney to a lesser extent. Increased RNA expression of HEMO was observed, for example, in ovary as well as kidney, esophagus, spleen, testis, lung, vagina, heart (left ventricle), and transformed fibroblasts to a lesser extent.

FIG. 1B depicts RNA expression levels (counts) of HHLA2 and HEMO in human cancers (TCGA datasets). For example, higher RNA expression levels of HHLA2 were observed in colon cancer and lung adenocarcinoma, while higher RNA expression levels of HEMO were observed in breast cancer, lung adenocarcinoma, and prostate cancer, as well as, to a lesser extent, in colon cancer.

FIG. 1C depicts HEMO gene expression relative to GAPDH of tumor tissues and histologically normal tissues adjacent to the tumor as assessed by real-time PCR. Samples were from a commercially available cDNA panel.

FIG. 1D depicts the expression of HEMO in human carcinoma cell lines. HEMO gene expression relative to GAPDH of human cancer cell lines as assessed by real-time PCR. MDA-MB-231 is a human triple-negative breast carcinoma cell line. SW480 and SW620 are human colon carcinoma cell lines; A549 and H460 are human lung carcinoma cell lines.

FIG. 1E shows that HEMO protein is expressed both on the cell surface and in the cytoplasm. Immune-fluorescent analysis of HEMO expression in the SW620 cell line is depicted, wherein cells were fixed and stained using either a HEMO or control antibody with or without permeabilizing the cell membrane and DAPI was used to stain the nuclei.

FIG. 1F shows the expression of HEMO protein expression in human tumor biopsies. Commercially available bladder, breast, and prostate tumor tissue microarrays were stained using a rabbit polyclonal HEMO antibody. Sections were counterstained using the DNA stain DAPI.

FIGS. 2A-2B depict the characterization of a commercially available HEMO antibody. (A) Western blot analysis of HEMO protein expression in the parental SW620 human colon carcinoma cell line, along with two clonally-derived SW620 cell lines in which HEMO protein expression was ‘knocked-out’ using a CRISPR-based strategy. (B) Western blot analysis of HEMO protein expression in the human breast carcinoma cell line MDA-MB-231 stably transfected with either a control plasmid (pCMV) or a plasmid encoding the full-length human HEMO protein. GAPDH is shown as a loading control.

FIG. 3 depicts cell-based binding of HEMO peptides to HLA-A2 in vitro. In particular, the graph shows in vitro binding of indicated concentrations of HEMO peptides designated in Table 1 to HLA-A2. The positive control peptide (WLLPGTSTV; SEQ ID NO: 101) is an agonist peptide derived from the tumor-associated antigen Brachyury, which is known to bind strongly to the HLA-A2 molecule. The negative control peptide (SYLIRALTL; SEQ ID NO: 102) is a peptide derived from influenza that binds strongly to HLA-A24 but not to HLA-A2.

FIG. 4 depicts the immune reactivity of 9-mer HEMO peptides. Specifically, the figure shows the identification of immune-reactive 9-mer HEMO peptides (identified in Table 1) in the blood of an HLA-A2—expressing prostate cancer patient.

FIG. 5 depicts cell-based binding of HHLA2 peptides to HLA-A2 in vitro. In particular, the graph shows in vitro binding of indicated concentrations of HHLA2 peptides designated in Table 13 to HLA-A2. The positive control peptide (WLLPGTSTV; SEQ ID NO: 101) is an agonist peptide derived from the tumor-associated antigen Brachyury, which is known to bind strongly to the HLA-A2 molecule. The negative control peptide (SYLIRALTL; SEQ ID NO: 102) is a peptide derived from influenza that binds strongly to HLA-A24 but not to HLA-A2.

FIG. 6 depicts a Western blot analysis of protein expression following vaccination of mice with an adenoviral vector encoding the full-length HEMO protein (Adeno-HEMO).

FIG. 7 depicts the results of an IFNγ-ELISPOT assay with 94 individual 15-mer peptides spanning the HEMO protein used as antigen for in vitro stimulation. Columns correspond to results with each of the 94 individual 15-mer HEMO peptides.

FIGS. 8A-8B are graphs depicting the anti-tumor effects of vaccination with HEMO peptides. Murine colon carcinoma MC38 cells were transfected to encode the full length HEMO protein. Mice bearing MC38-HEMO tumors were left untreated or treated with Adeno-HEMO vaccine, Adeno-HEMO plus anti-PD-L1, or Adeno-HEMO plus anti-PD-L1 and NHS-IL12 as indicated in the schema (FIG. 8A). FIG. 8B shows average tumor model in each group, wherein vaccine refers to Adeno-HEMO.

FIGS. 9A-9D further characterize the HEMO protein. FIG. 9A is a graphical representation of the region of homology shared between HEMO and other human proteins, which is located within the transmembrane region of HEMO. FIG. 9B is a representation of the regions of the HEMO protein used to design the overlapping 15-mer protein library used for immune assays. A total of 94 15-mer peptides are included in the mix. FIG. 9C is a heatmap representation of the number of antigen-dependent, cytokine-producing CD4+ and CD8+ T cells per 1×10\hat{ }5 CD4+ or CD8+ T cells following an overnight stimulation with a 15-mer overlapping HEMO peptide library (94-peptide mix). FIG. 9D is an example of a bladder cancer patient with a robust anti-HEMO immune response following the in-vitro stimulation as described above as assessed by the production of TNF and IFNγ as assessed by flow cytometry.

### DETAILED DESCRIPTION OF THE INVENTION

The invention provides human immunogenic epitopes of HEMO and HHLA2 human endogenous retroviruses (HERVs), which can be used in vaccines and other compositions for the prevention or therapeutic treatment cancer. In particular, the invention provides peptides, polypeptides, and proteins comprising, consisting essentially of, or consisting of the amino acid sequence of any one of SEQ ID NOs: 1-100 and 103. In a specific embodiment, the invention provides a peptide comprising, consisting essentially of, or consisting of the amino acid sequence of any one of SEQ ID NOs: 7-12, 20-28, 34-39, 53-59, 77-85, and 96-100.

In another aspect, the invention provides a polypeptide that comprises the HERV HEMO envelope (env) or fragment thereof, wherein one or more of the corresponding amino acid residues have been replaced with one or more of the enhancer agonist epitopes of SEQ ID NOs: 7-12, 20-28, and 34-39. In one embodiment, the invention provides a polypeptide that comprises the HERV HEMO env or fragment thereof, wherein one or more of the corresponding amino acid residues have been replaced with one or more of the enhancer agonist epitopes of SEQ ID NOs: 9, 11, 20-28, and 34-39.

In another aspect, the invention provides a polypeptide that comprises the HERV HHLA2 env or fragment thereof, wherein the corresponding amino acid residues have been replaced with one or more of the enhancer agonist epitopes of SEQ ID NOs: 53-59, 77-85, and 96-100. In one embodiment, the invention provides a polypeptide that comprises the HERV HHLA2 env or fragment thereof, wherein the corresponding amino acid residues have been replaced with one or more of the enhancer agonist epitopes of SEQ ID NOs: 54, 58, 59, 77-83, and 96-99.

A “polypeptide” is generally understood to be a linear organic polymer consisting of a large number of amino acid residues bonded together in a continuous, unbranched chain, forming part of, or the whole of, a protein molecule. A “peptide” is generally considered to be distinguished from a full-length protein or polypeptide on the basis of size, and, in one embodiment, as an arbitrary benchmark can be understood to contain approximately 50 or fewer amino acids, while polypeptides or full-length proteins are generally longer. However, the terms “peptide” and “polypeptide” can be used interchangeably in some embodiments to describe a protein useful in the present invention, or the term “protein” can be used generally.

The inventive peptide or polypeptide can be any suitable length. In one embodiment, a peptide of the invention has no more than 21 (e.g., no more than 20, no more than 19, no more than 18, no more than 17, no more than 16, no more than 15, no more than 14, no more than 13, no more than 12, no more than 11, or no more than 10) amino acid residues. The additional amino acid residues, if present, preferably are from the corresponding HERVs proteins (e.g., HERV-HEMO env or HERV-HHLA2 env). The additional amino acid residues can be positioned at either end or both ends of the amino acid sequence of SEQ ID NOs: 1-100 and 103.

A polypeptide for expression in a host cell, such as a yeast, is of a minimum size capable of being expressed recombinantly in the host cell. Accordingly, the polypeptide that is expressed by the host cell is preferably at least 25 amino acids in length, and is typically at least or greater than 25 amino acids in length, or at least or greater than 26 amino acids, at least or greater than 27 amino acids, at least or greater than 28 amino acids, at least or greater than 29 amino acids, at least or greater than 30 amino acids, at least or greater than 31 amino acids, at least or greater than 32 amino acids, at least or greater than 33 amino acids, at least or greater than 34 amino acids, at least or greater than 35 amino acids, at least or greater than 36 amino acids, at least or greater than 37 amino acids, at least or greater than 38 amino acids, at least or greater than 39 amino acids, at least or greater than 40 amino acids, at least or greater than 41 amino acids, at least or greater than 42 amino acids, at least or greater than 43 amino acids, at least or greater than 44 amino acids, at least or greater than 45 amino acids, at least or greater than 46 amino acids, at least or greater than 47 amino acids, at least or greater than 48 amino acids, at least or greater than 49 amino acids, or at least or greater than 50 amino acids in length, or at least 25-50 amino acids in length, at least 30-50 amino acids in length, or at least 35-50 amino acids in length, or at least 40-50 amino acids in length, or at least 45-50 amino acids in length, although smaller proteins may be expressed, and considerably larger proteins (e.g., hundreds of amino acids in length or even a few thousand amino acids in length) may be expressed.

Peptides and polypeptides (proteins) of the invention are, in some embodiments of the invention, used as antigens. According to the present invention, the general use herein of the term “antigen” refers to any portion of a protein (e.g., peptide, partial protein, full-length protein), wherein the protein is naturally occurring or synthetically derived or designed, to a cellular composition (whole cell, cell lysate or disrupted cells), to an organism (whole organism, lysate or disrupted cells), or to a carbohydrate, or other molecule, or a portion thereof. An antigen may elicit an antigen-specific immune response (e.g., a humoral and/or a cell-mediated immune response) against the same or similar antigens that are encountered in vitro, in vivo, or ex vivo by an element of the immune system (e.g., T cells, antibodies).

An antigen can be as small as a single epitope (e.g., SEQ ID NOs: 1-100 and 103 described herein), a single immunogenic domain or larger, and can include multiple epitopes or immunogenic domains. As such, the size of a protein antigen can be as small as about 8-11 amino acids (e.g., a peptide) and as large as a domain of a protein, a full-length protein, a multimer, a fusion protein, or a chimeric protein. Antigens useful in various immunotherapeutic compositions described herein include peptides, polypeptides, protein domain(s) (e.g., immunogenic domains), protein subunits, full-length proteins, multimers, fusion proteins, and chimeric proteins.

When referring to stimulation of an immune response, the term “immunogen” is a subset of the term “antigen” and, therefore, in some instances, can be used interchangeably with the term “antigen.” An immunogen, as used herein, describes an antigen which elicits a humoral and/or cell-mediated immune response (i.e., is immunogenic), such that administration of the immunogen to an individual mounts an antigen-specific immune response against the same or similar antigens that are encountered by the immune system of the individual. In one embodiment, the immunogen elicits a cell-mediated immune response, including a CD4⁺ T cell response (e.g., TH1, TH2, and/or TH17) and/or a CD8⁺ T cell response (e.g., a CTL response).

An “immunogenic domain” or “immunological domain” of a given protein (polypeptide) can be any portion, fragment or epitope of an antigen (e.g., a peptide fragment or subunit or an antibody epitope or other conformational epitope) that contains at least one epitope that can act as an immunogen when administered to an animal. Therefore, an immunogenic domain is larger than a single amino acid and is at least of a size sufficient to contain at least one epitope that can act as an immunogen. For example, a single protein can contain multiple different immunogenic domains. Immunogenic domains need not be linear sequences within a protein, such as in the case of a humoral immune response, where conformational domains are contemplated.

An epitope is defined herein as a single immunogenic site within a given antigen that is sufficient to elicit an immune response when provided to the immune system in the context of appropriate costimulatory signals and/or activated cells of the immune system. In other words, an epitope is the part of an antigen that is recognized by components of the immune system, and may also be referred to as an antigenic determinant. Those of skill in the art will recognize that T cell epitopes are different in size and composition from B cell or antibody epitopes, and that epitopes presented through the Class I MHC pathway differ in size and structural attributes from epitopes presented through the Class II MHC pathway. For example, T cell epitopes presented by Class I MHC molecules are typically between 8 and 11 amino acids in length, whereas epitopes presented by Class II MHC molecules are less restricted in length and may be up to 25 amino acids or longer. In addition, T cell epitopes have predicted structural characteristics depending on the specific MHC molecules bound by the epitope. Epitopes can be linear sequence epitopes or conformational epitopes (conserved binding regions). Most antibodies recognize conformational epitopes.

A “target antigen” is an antigen that is specifically targeted by an immunotherapeutic composition of the invention (i.e., an antigen, usually the native antigen, against which elicitation of an immune response is desired, even if the antigen used in the immunotherapeutic is an agonist of the native antigen). A “cancer antigen,” which also is referred to as a tumor-associated antigen (TAA), is an antigen that comprises at least one antigen that is associated with a cancer, such as an antigen expressed by a tumor cell, so that targeting the antigen also targets the tumor cell and/or cancer. A cancer antigen can include one or more antigens from one or more proteins, including one or more tumor-associated proteins.

Examples of antigens discovered in the present invention are provided herein (see Tables 1-3). A peptide, protein, or polypeptide useful in the present invention comprises, consists essentially of, or consists of at least one of peptides represented by SEQ ID NOs: 1-100 and 103. However, other epitopes can be additionally included in an antigen for use in the present invention.

In addition, a HERVs antigen useful in the present invention may include one or more additional amino acid mutations (substitutions, insertions or deletions), for example, to inactivate or delete a natural biological function of the native protein (e.g., to improve expression or enhance safety of the antigen).

The peptide or polypeptide (protein) of the invention can be prepared by any method, such as by synthesizing the peptide or by expressing a nucleic acid encoding an appropriate amino acid sequence for the peptide or polypeptide in a cell and, in some embodiments, harvesting the peptide or polypeptide from the cell. In some embodiments, the peptide or polypeptide is not harvested from the cell, such as in embodiments of the invention directed to a yeast-based immunotherapy composition, which is described in detail below. A combination of such methods of production of peptides and polypeptides also can be used. Methods of de novo synthesizing peptides and methods of recombinantly producing peptides or polypeptides are known in the art

The invention also provides a nucleic acid molecule comprising a nucleic acid sequence encoding the peptide or the polypeptide. The nucleic acid molecule can comprise DNA (genomic or cDNA) or RNA, and can be single or double stranded. Furthermore, the nucleic acid molecule can comprise nucleotide analogues or derivatives (e.g., inosine or phophorothioate nucleotides and the like). The nucleic acid sequence can encode the peptide or polypeptide alone or as part of a fusion protein. The nucleic acid sequence encoding the peptide or polypeptide can be provided as part of a construct comprising the nucleic acid molecule and elements that enable delivery of the nucleic acid molecule to a cell, and/or expression of the nucleic acid molecule in a cell. Such elements include, for example, expression vectors, promoters, and transcription and/or translation control sequences. Such constructs can also be referred to as “recombinant nucleic acid molecules.” Suitable vectors, promoters, transcription/translation sequences, and other elements, as well as methods of preparing such nucleic acid molecules and constructs, are known in the art. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a peptide or polypeptide. Similarly, the phrase “recombinant nucleic acid molecule” primarily refers to a nucleic acid molecule operatively linked to an element such as a transcription control sequence, but can be used interchangeably with the phrase “nucleic acid molecule.”

The invention further provides a vector comprising the nucleic acid molecule comprising a nucleic acid sequence encoding the peptide or the polypeptide (e.g., full-length HEMO protein). Examples of suitable vectors include plasmids (e.g., DNA plasmids), bacteria, yeast, listeria, and viral vectors, such as poxvirus, retrovirus, adenovirus, adeno-associated virus, herpes virus, polio virus, alphavirus, baculorvirus, and Sindbis virus.

In a first aspect, the vector is a plasmid (e.g., DNA plasmid). The plasmid can be complexed with chitosan.

In a second aspect, the vector is a poxvirus (e.g., chordopox virus vectors and entomopox virus vectors). Suitable poxviruses include orthopox, avipox, parapox, yatapox, and molluscipox, raccoon pox, rabbit pox, capripox (e.g., sheep pox), leporipox, and suipox (e.g., swinepox). Examples of avipox viruses include fowlpox, pigeonpox, canarypox, such as ALVAC, mynahpox, uncopox, quailpox, peacockpox, penguinpox, sparrowpox, starlingpox, and turkeypox. Examples of orthopox viruses include smallpox (also known as variola), cowpox, monkeypox, vaccinia, ectromelia, camelpox, raccoonpox, and derivatives thereof.

The term “vaccinia virus” refers to both the wild-type vaccinia virus and any of the various attenuated strains or isolates subsequently isolated including, for example, modified vaccinia Ankara (MVA), NYVAC, TROYVAC, Dry-Vax (also known as vaccinia virus-Wyeth), PDXVAC-TC (Schering-Plough Corporation), vaccinia virus-Western Reserve, vaccinia virus-EM63, vaccinia virus-Lister, vaccinia virus-New York City Board of Health, vaccinia virus-Temple of Heaven, vaccinia virus-Copenhagen, ACAM1000, ACAM2000, and modified vaccinia virus Ankara-Bavarian Nordic (“MVA-BN”).

In certain embodiments, the MVA is selected from the group consisting of MVA-572, deposited at the European Collection of Animal Cell Cultures (“ECACC”), Health Protection Agency, Microbiology Services, Porton Down, Salisbury SP4 OJG, United Kingdom (“UK”), under the deposit number ECACC 94012707 on Jan. 27, 1994; MVA-575, deposited at the ECACC under deposit number ECACC 00120707 on Dec. 7, 2000; MVA-Bavarian Nordic (“MVA-BN”), deposited at the ECACC under deposit number V00080038 on Aug. 30, 2000; and derivatives of MVA-BN. Additional exemplary poxvirus vectors are described in U.S. Pat. No. 7,211,432.

The vaccinia virus MVA was generated by 516 serial passages on chicken embryo fibroblasts of the Ankara strain of Vaccinia virus, referred to as chorioallantois virus Ankara (CVA) (see Mayr et al., Infection, 3: 6-14 (1975)). The genome of the resulting attenuated MVA lacks approximately 31 kilobase pairs of genomic DNA compared to the parental CVA strain and is highly host-cell restricted to avian cells (see Meyer et al., J. Gen. Virol., 72: 1031-1038 (1991)). It was shown in a variety of animal models that the resulting MVA was significantly avirulent (Mayr et al., Dev. Biol. Stand., 41: 225-34 (1978)). This MVA strain has been tested in clinical trials as a vaccine to immunize against smallpox in humans (see Mary et al., Zbl. Bakt. Hyg. I, Abt. Org. B, 167: 375-390 (1987); and Stickl et al., Dtsch. Med. Wschr., 99: 2386-2392 (1974)). Those studies involved over 120,000 humans, including high-risk patients, and proved that compared to vaccinia virus-based vaccines, MVA had diminished virulence or infectiousness while still able to induce a good specific immune response. Although MVA-BN is preferred for its better safety profile because it is less replication competent than other MVA strains, all MVAs are suitable for this invention, including MVA-BN and its derivatives.

Both MVA and MVA-BN are able to efficiently replicate their DNA in mammalian cells even though they are avirulent. This trait is the result of losing two important host range genes among at least 25 additional mutations and deletions that occurred during its passages through chicken embryo fibroblasts (see Meyer et al., Gen. Virol., 72: 1031-1038 (1991); and Antoine et al., Virol., 244: 365-396 (1998)). In contrast to the attenuated Copenhagen strain (NYVAC) and host range restricted avipox (ALVAC), both-early and late transcription in MVA are unimpaired, which allows for continuous gene expression throughout the viral life cycle (see Sutter et al., Proc. Nat'l Acad. Sci. USA, 89: 10847-10851 (1992)). In addition, MVA can be used in conditions of pre-existing poxvirus immunity (Ramirez et al., J. Virol., 74: 7651-7655 (2000)).

Both MVA and MVA-BN lack approximately 15% (31 kb from six regions) of the genome compared with the ancestral chorioallantois vaccinia virus Ankara (“CVA”). The deletions affect a number of virulence and host range genes, as well as the gene for Type A inclusion bodies. MVA-BN can attach to and enter human cells where virally-encoded genes are expressed very efficiently. However, assembly and release of progeny virus does not occur. MVA-BN is strongly adapted to primary chicken embryo fibroblast (CEF) cells and does not replicate in human cells. In human cells, viral genes are expressed, and no infectious virus is produced. Despite its high attenuation and reduced virulence, in preclinical studies, MVA-BN has been shown to elicit both humoral and cellular immune responses to vaccinia and to heterologous gene products encoded by genes cloned into the MVA genome (see Harrer et al., Antivir. Ther., 10(2): 285-300 (2005); Cosma et al., Vaccine, 22(1): 21-29 (2003); Di Nicola et al., Hum. Gene Ther., 14(14): 1347-1360 (2003); and Di Nicola et al., Clin. Cancer Res., 10(16): 5381-5390 (2004)).

The reproductive replication of a virus is typically expressed by the amplification ratio. The term “amplification ratio” refers to the ratio of virus produced from an infected cell (“output”) to the amount originally used to infect the cells in the first place (“input”). An amplification ratio of “1” defines an amplification status in which the amount of virus produced from infected cells is the same as the amount initially used to infect the cells, which means that the infected cells are permissive for virus infection and reproduction. An amplification ratio of less than 1 means that infected cells produce less virus than the amount used to infect the cells in the first place, and indicates that the virus lacks the capability of reproductive replication, which is a measure of virus attenuation.

Thus, the term “not capable of reproductive replication” means that an MVA or MVA derivative has an amplification ratio of less than 1 in one or more human cell lines, such as, for example, the human embryonic kidney 293 cell line (HEK293, which is deposited under deposit number ECACC No. 85120602), the human bone osteosarcoma cell line 143B (deposited under deposit number ECACC No. 91112502), the human cervix adenocarcinoma cell line HeLa (deposited at the American Type Culture Collection (ATTC) under deposit number ATCC No. CCL-2), and the human keratinocyte cell line HaCat (see Boukamp et al., J. Cell Biol., 106(3): 761-71 (1988)).

MVA-BN does not reproductively replicate in the human cell lines HEK293, 143B, HeLa, and HaCat (see U.S. Pat. Nos. 6,761,893 and 6,193,752, and International Patent Application Publication No. WO 2002/042480). For example, in one exemplary experiment, MVA-BN exhibited an amplification ratio of 0.05 to 0.2 in HEK293 cells, an amplification ratio of 0.0 to 0.6 in 143B cells, an amplification ratio of 0.04 to 0.8 in HeLa cells, and an amplification ratio of 0.02 to 0.8 in HaCat cells. Thus, MVA-BN does not reproductively replicate in any of the human cell lines HEK293, 143B, HeLa, and HaCat. In contrast, the amplification ratio of MVA-BN is greater than 1 in primary cultures of chicken embryo fibroblast cells (CEF) and in baby hamster kidney cells (BHK, which is deposited under deposit number ATCC No. CRL-1632). Therefore MVA-BN can easily be propagated and amplified in CEF primary cultures with an amplification ratio above 500, and in BHK cells with an amplification ratio above 50.

As noted above, all MVAs are suitable for this invention, including MVA-BN and its derivatives. The term “derivatives” refers to viruses showing essentially the same replication characteristics as the strain deposited with ECACC on Aug. 30, 2000, under deposit number ECACC No. V00080038 but showing differences in one or more parts of its genome. Viruses having the same “replication characteristics” as the deposited virus are viruses that replicate with similar amplification ratios as the deposited strain in CEF cells, in BHK cells, and in the human cell lines HEK293, 143B, HeLa, and HaCat.

When the vector is for administration to a subject (e.g., human), the vector (e.g., poxvirus) preferably has a low replicative efficiency in a target cell (e.g., no more than about 1 progeny per cell or, more preferably, no more than 0.1 progeny per cell are produced). Replication efficiency can readily be determined empirically by determining the virus titer after infection of the target cell.

In addition to the nucleic acid molecule encoding the polypeptide (protein) or polypeptide (i.e., the peptide or polypeptide comprising, consisting essentially of, or consisting of at least one HERVs epitope described herein), a vector useful in the invention (e.g., a plasmid or a viral vector) also can comprise a nucleic acid sequence encoding one or more immunostimulatory/regulatory molecules, granulocyte macrophage colony stimulating factor (GM-CSF), cytokines, and/or molecules that can enhance an immune response (e.g., additional tumor-associated antigens). Exemplary additional tumor-associated antigens (TAAs, also referred to as cancer antigens) include, but are not limited to, 5-α-reductase, α-fetoprotein (AFP), AM-1, APC, April, B melanoma antigen gene (BAGE), β-catenin, Bcl12, bcr-abl, Brachyury, CA-125, caspase-8 (CASP-8 also known as FLICE), Cathepsins, CD19, CD20, CD21/complement receptor 2 (CR2), CD22/BL-CAM, CD23/FccRII, CD33, CD35/complement receptor 1 (CR1), CD44/PGP-1, CD45/1eucocyte common antigen (LCA), CD46/membrane cofactor protein (MCP), CD52/CAMPATH-1, CD55/decay accelerating factor (DAF), CD59/protectin, CDC27, CDK4, carcinoembryonic antigen (CEA), c-myc, cyclooxygenase-2 (cox-2), deleted in colorectal cancer gene (DCC), DcR3, E6/E7, CGFR, EMBP, Dna78, farnesyl transferase, fibroblast growth factor-8a (FGF8a), fibroblast growth factor-8b (FGF8b), FLK-1/KDR, folic acid receptor, G250, G melanoma antigen gene family (GAGE-family), gastrin 17, gastrin-releasing hormone, ganglioside 2 (GD2)/ganglioside 3 (GD3)/ganglioside-monosialic acid-2 (GM2), gonadotropin releasing hormone (GnRH), UDP-GlcNAc:R₁Man(α1-6)R₂ [GlcNAc to Man(α1-6)] β1,6-N-acetylglucosaminyltransferase V (GnT V), GP1, gp100/Pme117, gp-100-in4, gp15, gp75/tyrosine-related protein-1 (gp75/TRP-1), human chorionic gonadotropin (hCG), heparanase, Her2/neu, human mammary tumor virus (HMTV), 70 kiloDalton heat-shock protein (HSP70), human telomerase reverse transcriptase (hTERT), insulin-like growth factor receptor-1 (IGFR-1), interleukin-13 receptor (IL-13R), inducible nitric oxide synthase (iNOS), Ki67, KIAA0205, K-ras, H-ras, N-ras, KSA, LKLR-FUT, melanoma antigen-encoding family (MAGE-family, including at least MAGE-1, MAGE-2, MAGE-3, and MAGE-4), mammaglobin, MAP17, Melan-A/melanoma antigen recognized by T-cells-1 (MART-1), mesothelin, MIC A/B, MT-MMPs, mucin (e.g., MUC1), testes-specific antigen NY-ESO-1, osteonectin, p15, P170/MDR1, p53, p97/melanotransferrin, PAI-1, platelet-derived growth factor (PDGF), μPA, PRAME, probasin, progenipoietin, prostate-specific antigen (PSA), prostate-specific membrane antigen (PSMA), RAGE-1, Rb, RCAS1, mutated Ras, SART-1, SSX-family, STAT3, STn, TAG-72, transforming growth factor-alpha (TGF-α), transforming growth factor-beta (TGF-β), Thymosin-beta-15, tumor necrosis factor-alpha (TNF-α), TP1, TRP-2, tyrosinase, vascular endothelial growth factor (VEGF), ZAG, p16INK4, and glutathione-S-transferase (GST), as well as modified versions thereof (e.g., CEA-6D).

In the case of a viral vector, the nucleic acid encoding the peptide, as well as any other exogenous gene(s), preferably are inserted into a site or region (insertion region) in the vector (e.g., poxvirus) that does not affect virus viability of the resultant recombinant virus. Such regions can be readily identified by testing segments of virus DNA for regions that allow recombinant formation without seriously affecting virus viability of the recombinant virus.

The thymidine kinase (TK) gene is an insertion region that can readily be used and is present in many viruses. In particular, the TK gene has been found in all examined poxvirus genomes. Additional suitable insertion sites are described in International Patent Application Publication WO 2005/048957. For example, in fowlpox, insertion regions include, but are not limited to, the BamHI J fragment, EcoRI-HindIII fragment, BamHI fragment, EcoRV-HindIII fragment, long unique sequence (LUS) insertion sites (e.g., FPV006/FPV007 and FPV254/FPV255), FP14 insertion site (FPV060/FPV061), and 43K insertion site (FPV107/FPV108). In vaccinia, insertion sites include, but are not limited to, 44/45, 49/50, and 124/125.

When the vector is a recombinant fowlpox virus comprising a nucleic acid encoding the peptide and/or other exogenous gene(s) (e.g., encoding one or more immunostimulatory/regulatory molecules), the nucleic acid encoding the peptide can be inserted in one region (e.g., the FP14 region), and the exogenous gene(s) can be inserted in another region (e.g., the BamHI J region).

The inventive vector can include suitable promoters and regulatory elements, such as a transcriptional regulatory element or an enhancer. Suitable promoters include the SV40 early promoter, an RSV promoter, the retrovirus LTR, the adenovirus major late promoter, the human CMV immediate early I promoter, and various poxvirus promoters, such as the Pr7.5K promoter, 30K promoter, 40K promoter, 13 promoter, Prs promoter, PrsSynIIm promoter, PrLE1 promoter, synthetic early/late (sE/L) promoter, HH promoter, 11K promoter, and Pi promoter. While the promoters typically will be constitutive promoters, inducible promoters also can be used in the inventive vectors. Such inducible systems allow regulation of gene expression.

In one aspect of the invention, a cell (e.g., isolated cell) comprising (1) the peptide or polypeptide, (2) a nucleic acid molecule encoding the peptide or polypeptide, and/or (3) a vector comprising the nucleic acid molecule also is provided herein. Suitable cells include prokaryotic and eukaryotic cells, e.g., mammalian cells, yeast, fungi other than yeast, and bacteria (such as E. coli). The cell can be used in vitro, such as for research or for production of the peptide or polypeptide, or the cell can be used in vivo. In one embodiment, the cell is a yeast cell, which may be used to provide a yeast vehicle component of the yeast-based immunotherapy composition as described herein. In another embodiment, the cell can be a peptide-pulsed antigen presenting cell. Suitable antigen presenting cells include, but are not limited to, dendritic cells, B lymphocytes, monocytes, macrophages, and the like.

In one embodiment, the cell is dendritic cell. Dendritic cells of different maturation stages can be isolated based on the cell surface expression markers. For example, mature dendritic cells are less able to capture new proteins for presentation but are much better at stimulating resting T cells to grow and differentiate. Thus, mature dendritic cells can be of importance. Mature dendritic cells can be identified by their change in morphology and by the presence of various markers. Such markers include, but are not limited to, cell surface markers such as B7.1, B7.2, CD40, CD11, CD83, and MHC class II. Alternatively, maturation can be identified by observing or measuring the production of pro-inflammatory cytokines.

Dendritic cells can be collected and analyzed using typical cytofluorography and cell sorting techniques and devices, such as a fluorescence-activated cell sorter (FACS). Antibodies specific to cell surface antigens of different stages of dendritic cell maturation are commercially available.

The peptide, polypeptide, nucleic acid, vector, or cell can be isolated. The term “isolated” as used herein encompasses compounds or compositions that have been removed from a biological environment (e.g., a cell, tissue, culture medium, body fluid, etc.) or otherwise increased in purity to any degree (e.g., isolated from a synthesis medium). Isolated compounds and compositions, thus, can be synthetic or naturally produced.

The peptide, polypeptide, nucleic acid, vector, or cell can be formulated as a composition (e.g., pharmaceutical composition) comprising the peptide, polypeptide, nucleic acid, vector, or cell and a carrier (e.g., a pharmaceutically or physiologically acceptable carrier). Furthermore, the peptide, polypeptide, nucleic acid, vector, cell, or composition of the invention can be used in the methods described herein alone or as part of a pharmaceutical formulation.

The composition (e.g., pharmaceutical composition) can comprise more than one peptide, polypeptide, nucleic acid, vector, or cell of the invention. Vectors and compositions of the invention can further include or can be administered with (concurrently, sequentially, or intermittently with) any other agents or compositions or protocols that are useful for inhibiting, preventing, or treating cancer or any compounds that treat or ameliorate any symptom of cancer. For example, the composition can comprise one or more other pharmaceutically active agents or drugs. Examples of such other pharmaceutically active agents or drugs that may be suitable for use in the pharmaceutical composition include anticancer agents (e.g., chemotherapeutic or radiotherapeutic agents), antimetabolites, hormones, hormone antagonists, antibiotics, antiviral drugs, antifungal drugs, cyclophosphamide, and combinations thereof. Suitable anticancer agents include, without limitation, alkylating agents, folate antagonists, purine antagonists, pyrimidine antagonists, spindle poisons, topoisomerase inhibitors, apoptosis inducing agents, angiogenesis inhibitors, podophyllotoxins, nitrosoureas, cisplatin, carboplatin, interferon, asparginase, tamoxifen, leuprolide, flutamide, megestrol, mitomycin, bleomycin, doxorubicin, irinotecan, taxol, geldanamycin (e.g., 17-AAG), and various anti-cancer peptides and antibodies known in the art.

Exemplary alkylating agents include, but are not limited to, nitrogen mustards (e.g., mechlorethamine, cyclophosphamide, melphalan, uracil mustard, or chlorambucil), alkyl sulfonates (e.g., busulfan), nitrosoureas (e.g., carmustine, lomustine, semustine, streptozocin, or dacarbazine). Exemplary antimetabolites include, but are not limited to, folic acid analogs (e.g., methotrexate), pyrimidine analogs (e.g., 5-fluorouracil (5-FU) or cytarabine), and purine analogs (e.g., mercaptopurine or thioguanine). Exemplary hormones and hormone antagonists include, but are not limited to, adrenocorticosteroids (e.g., prednisone), progestins (e.g., hydroxyprogesterone caproate, medroxyprogesterone acetate, and magestrol acetate), estrogens (e.g., diethylstilbestrol and ethinyl estradiol), antiestrogens (e.g., tamoxifen), and androgens (e.g., testosterone proprionate and fluoxymesterone). Other exemplary agents include, but are not limited to, vinca alkaloids (e.g., vinblastine, vincristine, or vindesine), epipodophyllotoxins (e.g., etoposide or teniposide), antibiotics (e.g., dactinomycin, daunorubicin, doxorubicin, bleomycin, plicamycin, or mitocycin C), enzymes (e.g., L-asparaginase), platinum coordination complexes (e.g., cis-diamine-dichloroplatinum II also known as cisplatin), substituted ureas (e.g., hydroxyurea), methyl hydrazine derivatives (e.g., procarbazine), and adrenocortical suppressants (e.g., mitotane and aminoglutethimide).

Chemotherapeutics that can be concurrently, sequentially or intermittently administered with the vectors and compositions disclosed herein include Adriamycin, Alkeran, Ara-C, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin, Enzalutamide (MDV-3100 or XTANDI™), and calcitriol. Exemplary immunomodulators and/or cytokines include, but are not limited to, AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, tumor necrosis factor (TNF)-α, and TNF-β.

Other agents, compositions or protocols (e.g., therapeutic protocols) that are useful for the treatment of cancer in conjunction with the peptides, polypeptides (proteins), nucleic acids, vectors, cells, and compositions of the invention include, but are not limited to, surgical resection of a tumor, radiation therapy, allogeneic or autologous stem cell transplantation, T cell adoptive transfer, and/or targeted cancer therapies (e.g., small molecule drugs, biologics, or monoclonal antibody therapies that specifically target molecules involved in tumor growth and progression, including, but not limited to, selective estrogen receptor modulators (SERMs), aromatase inhibitors, tyrosine kinase inhibitors, serine/threonine kinase inhibitors, histone deacetylase (HDAC) inhibitors, retinoid receptor activators, apoptosis stimulators, angiogenesis inhibitors, poly (ADP-ribose) polymerase (PARP) inhibitors, or immunostimulators). Additionally, or alternatively, the agent can be a cancer vaccine, such as PANVAC, PROSTVAC, MVA-Brachyury TRICOM, yeast-Brachyury, AdCEA Avelumab (Avel) Folfox, CEA-MUC-TRICOM CV301, or Bacillus Calmette-Guerin (BCG) alone or combined with PANVAC.

The additional active agent (e.g., chemotherapeutic agent) can be administered before, concurrently with (including simultaneously), alternating with, sequentially, or after administration with the vectors and compositions disclosed herein. In certain embodiments, one or more (e.g., 2, 3, 4, or 5) chemotherapeutic agents is administered in combination with the vectors and compositions disclosed herein.

The additional active agent can be administered alone or in a composition. The additional active agent can be formulated by inclusion in a vector (e.g., plasmid or viral vector), in liposomes (tecemotide, which is also known as STIMUVAX™, L-BLP25, or BLP25 liposome vaccine), or in nanoparticles (e.g., VERSAMUNE™ nanotechnology).

The carrier can be any of those conventionally used and is limited only by physio-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular peptide, polypeptide, nucleic acid, vector, cell, or composition thereof of the invention and other active agents or drugs used, as well as by the particular method used to administer the peptide, polypeptide, nucleic acid, vector, cell, or composition thereof.

The composition additionally or alternatively can comprise one or more immunostimulatory/regulatory molecules. Any suitable immunostimulatory/regulatory molecule can be used, such as interleukin (IL)-2, IL-4, IL-6, IL-12, IL-15, IL-15/IL-15Ra, IL-15/IL-15Ra-Fc, interferon (IFN)-γ, tumor necrosis factor (TNF)-α, B7.1, B7.2, ICAM-1, ICAM-2, LFA-1, LFA-2, LFA-3, CD70, CD-72, RANTES, G-CSF, GM-CSF, OX-40L, 41 BBL, anti-CTLA-4, IDO inhibitor, anti-PDL1, anti-PD1, and combinations thereof. In one embodiment, the IL-12 is NHS-IL12, which is an immunocytokine composed of two IL-12 heterodimers fused to the NHS76 antibody (see Strauss et al., Clinical Cancer Research, 25(1): 99-109 (2019)). Preferably, the composition comprises a combination of B7.1, ICAM-1, and LFA-3 (also referred to as TRICOM). The one or more immunostimulatory/regulatory molecules can be administered in the form of a vector (e.g., a recombinant viral vector, such as a poxvirus vector) comprising a nucleic acid encoding one or more immunostimulatory/regulatory molecules. For example, the one or more immunostimulatory/regulatory molecules (e.g., IL-12) can be administered in the form of a DNA plasmid with or without chitosan. Alternatively, the one or more immunostimulatory/regulatory molecules can be administered as a protein (e.g., recombinant protein), such as a protein (e.g., recombinant IL-12) admixed with chitosan. One or more immunostimulatory/regulatory molecules also can be administered in combination with, or concurrently with, a yeast-based immunotherapy composition of the invention.

In one embodiment of the invention, the composition comprises a first recombinant vector comprising the nucleic acid encoding the inventive peptide or polypeptide (protein) and second recombinant vector comprising a nucleic acid encoding B7.1, ICAM-1, and LFA-3 (TRICOM). In another embodiment, the nucleic acid encoding the inventive peptide or polypeptide (protein) and the nucleic acid encoding B7.1, ICAM-1, and LFA-3 are in the same recombinant vector. The first and/or second vectors additionally can comprise a nucleic acid encoding another tumor associated antigen (e.g., CEA, MUC1, PSA, and/or Brachyury), a modified version thereof (e.g., CEA-6D), or an epitope thereof.

For example, the recombinant vector can be an avipox vector (e.g., canarypox virus or a fowlpox virus) comprising the nucleic acid encoding the inventive peptide and nucleic acids encoding a B7-1 polypeptide, an ICAM-1 polypeptide, and an LFA-3 polypeptide. Alternatively, the recombinant vector can be an orthopox virus comprising the nucleic acid encoding the inventive peptide and nucleic acids encoding a B7-1 polypeptide, an ICAM-1 polypeptide, and an LFA-3 polypeptide.

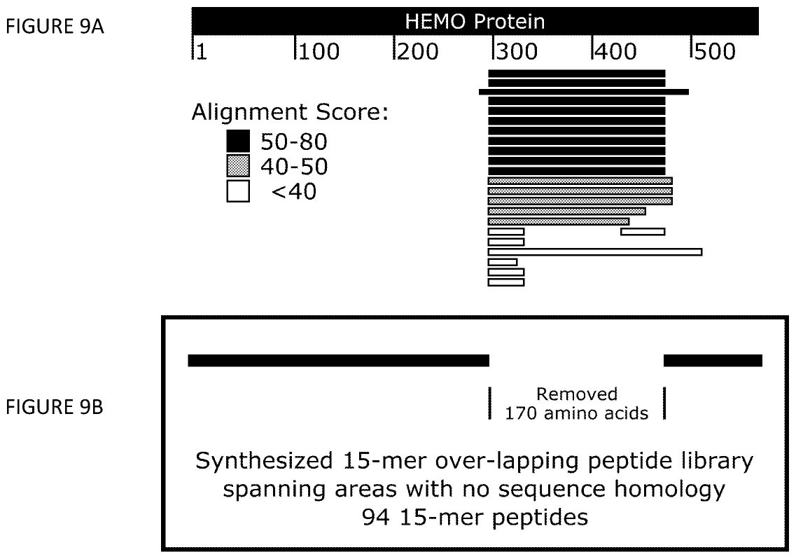

In one aspect of the invention, the composition comprises a vector comprising the nucleic acid molecule comprising a nucleic acid sequence encoding the peptide or the polypeptide (e.g., full-length HEMO protein) and one or both of anti-PD-L1 and NHS-IL12 (see FIGS. 8A-8B).

The invention provides a method of transducing dendritic cells with the peptide, polypeptide, nucleic acid, vector, cell, or composition thereof, and optionally immunostimulatory/regulatory molecules, such as for example, B7-1, ICAM-1 and LFA-3 (TRICOM). In one aspect of the invention, dendritic cells transduced with the peptide, polypeptide, nucleic acid, vector, cell, or composition thereof are administered to the host generate an immune response, such as activation of a cytotoxic T cell response.

The invention provides methods of treating a subject suffering from or susceptible to a tumor and/or enhancing an immune response against cancer and/or inhibiting a cancer. In a first embodiment, the inventive methods comprise administering a therapeutically effective amount of one or more of the peptide, polypeptide, nucleic acid, vector, cell, or composition thereof to a subject. The inventive peptide, polypeptide, nucleic acid, vector, cell, or composition thereof can be used to prevent the development of cancer, particularly in an individual at higher risk to develop such cancer than other individuals, or to treat a patient afflicted with cancer. The inventive peptide, polypeptide, nucleic acid, vector, cell, or composition thereof is useful for preventing emergence of cancer, arresting progression of cancer or eliminating cancer. More particularly, the inventive peptide, polypeptide, nucleic acid, vector, cell, or composition thereof can be used to prevent, inhibit or delay the development of tumors, and/or to prevent, inhibit or delay tumor migration and/or tumor invasion of other tissues (metastases) and/or to generally prevent or inhibit progression of cancer in an individual. The inventive peptide, polypeptide, nucleic acid, vector, cell, or composition thereof can also be used to ameliorate at least one symptom of the cancer, such as by reducing tumor burden in the individual; inhibiting tumor growth in the individual; increasing survival of the individual; and/or preventing, inhibiting, reversing or delaying progression of the cancer in the individual. The inventive peptide, polypeptide, nucleic acid, vector, cell, or composition thereof can be used to treat a subject with any stage of cancer.

The inventive methods can comprise obtaining (by isolating) dendritic cells from a subject, treating the dendritic cells with one or more of the therapeutically effective amount of the peptide, polypeptide, nucleic acid, vector, cell, or composition thereof, and administering the treated dendritic cells to the subject.

The inventive methods can comprise (a) obtaining (isolating) peripheral blood mononuclear cells (PBMCs) from a subject, (b) isolating dendritic cells from the PBMCs, (c) treating the dendritic cells with one or more of the therapeutically effective amount of the peptide, polypeptide, nucleic acid, vector, cell, or composition thereof ex vivo, (d) activating the PBMCs with the treated dendritic cells ex vivo, and (e) administering the activated PBMCs to the subject.

The inventive methods also can comprise a method for inhibiting cancer in a subject comprising (a) obtaining (isolating) PBMCs from a subject, (b) isolating dendritic cells from the PBMCs, (c) treating the dendritic cells with one or more of the therapeutically effective amount of the peptide, polypeptide, nucleic acid, vector, cell, or composition thereof ex vivo, (d) activating the PBMCs with the treated dendritic cells ex vivo, (e) isolating T lymphocytes from the activated PBMCs ex vivo, and (f) administering the isolated T lymphocytes to the subject.

The invention also provides the use of adoptively transferred T cells stimulated in vitro with one or more of the therapeutically effective amount of the peptide, polypeptide, nucleic acid, vector, cell, or composition thereof to inhibit cancer in a subject.

The invention also provides T-cell receptor (TCR) engineered T cells and TCR engineered NK cells for treating (e.g., inhibiting) cancer in a subject. TCR engineered T cells and TCR engineered NK cells can be prepared by any suitable methods, such as those described in Ping et al., Protein Cell, 9(3) 254-266 (2018). In one embodiment, the TCR engineered T cells and TCR engineered NK cells target cancer cells expressing HEMO and/or HHLA2 HERVS, such as the epitopes of SEQ ID NOs: 1-100 and 103.

Treatment (e.g., inhibiting cancer and/or enhancing an immune response against cancer) comprises, but is not limited to, destroying tumor cells, reducing tumor burden, inhibiting tumor growth, reducing the size of the primary tumor, reducing the number of metastatic legions, increasing survival of the individual, delaying, inhibiting, arresting or preventing the onset or development of metastatic cancer (such as by delaying, inhibiting, arresting or preventing the onset of development of tumor migration and/or tumor invasion of tissues outside of primary cancer and/or other processes associated with metastatic progression of cancer), delaying or arresting primary cancer progression, improving immune responses against the tumor, improving long term memory immune responses against the tumor antigens, and/or improving the general health of the individual. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

The cancer can be any cancer, including, but not limited to, cancer of the head and neck, eye, skin, mouth, throat, esophagus, chest, bone, lung, urethra, uterine, bladder, colon, sigmoid, rectum, stomach, prostate, breast, ovaries, kidney, liver, pancreas, brain, intestine, fallopian tube, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, medulloblastoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acutenonlymphocyctic leukemia, acute undifferentiated leukemia, chronic myelocytic leukemia, chronic lymphocytic leukemia, hairy cell leukemia, or multiple myeloma.

The peptide, polypeptide, nucleic acid, vector, cell, or composition thereof can be administered to the subject by any method. For example, the peptide, polypeptide, or nucleic acid encoding the peptide or polypeptide (e.g., as a vector) can be introduced into a cell (e.g., in a host) by any of various techniques, such as by contacting the cell with the peptide, polypeptide, the nucleic acid, or a composition comprising the nucleic acid as part of a construct, as described herein, that enables the delivery and expression of the nucleic acid. Specific protocols for introducing and expressing nucleic acids in cells are known in the art.

Suitable methods of administering peptides, polypeptides (proteins), nucleic acids, vectors, cells, and compositions to hosts (subjects) are known in the art. The host (subject or individual) can be any suitable host, such as a mammal (e.g., a rodent, such as a mouse, rat, hamster, or guinea pig, rabbit, cat, dog, pig, goat, cow, horse, primate, or human).

For example, the peptide, polypeptide, nucleic acid, or vector (e.g., recombinant poxvirus) can be administered to a host by exposure of tumor cells to the peptide, polypeptide, nucleic acid, or vector ex vivo or by injection of the peptide, polypeptide, nucleic acid, or vector into the host. The peptide, polypeptide, nucleic acid, vector (e.g., recombinant poxvirus) or combination of vectors, cell, and composition can be directly administered (e.g., locally administered) by direct injection into the cancerous lesion or tumor or by topical application (e.g., with a pharmaceutically acceptable carrier).

The peptide, polypeptide, nucleic acid, vector, cell, or composition thereof can be administered alone or in combination with adjuvants, incorporated into liposomes (as described in, e.g., U.S. Pat. Nos. 5,643,599, 5,464,630, 5,059,421, and 4,885,172), incorporated into nanoparticles (e.g., VERSAMUNE™ nanotechnology), administered with cytokines, administered with biological response modifiers (e.g., interferon, interleukin-2 (IL-2), administered colony-stimulating factors (CSF, GM-CSF, and G-CSF), and/or administered other reagents in the art that are known to enhance immune response.

Examples of suitable adjuvants include alum, aluminum salts, aluminum phosphate, aluminum hydroxide, aluminum silica, calcium phosphate, incomplete Freund's adjuvant, saponins, such as QS21 (an immunological adjuvant derived from the bark of the South American tree Quillaja saponaria Molina), monophosphoryl lipid A (MLP-A), and RIBI DETOX™ adjuvant.

In one aspect, the adjuvant for use in the invention is the cytokine GM-CSF. GM-CSF has been shown to be an effective vaccine adjuvant because it enhances antigen processing and presentation by dendritic cells. Experimental and clinical studies suggest that recombinant GM-CSF can boost host immunity directed at a variety of immunogens.

GM-CSF can be administered using a viral vector (e.g., poxvirus vector) or as an isolated protein in a pharmaceutical formulation. GM-CSF can be administered to the host before, during, or after the initial administration of the peptide, polypeptide, nucleic acid, vector, cell, or composition thereof to enhance the antigen-specific immune response in the host. For example, recombinant GM-CSF protein can be administered to the host on each day of vaccination with the peptide, polypeptide, nucleic acid, vector, cell, or composition thereof and for each of the following 3 days (i.e. a total of 4 days). Any suitable dose of GM-CSF can be used. For instance, 50-500 μg (e.g., 100 μg, 200 μg, 300 μg, 400 μg, and ranges therebetween) of recombinant GM-CSF can be administered per day. The GM-CSF can be administered by any suitable method (e.g., subcutaneously) and, preferably, is administered at or near the site of the vaccination of a host with the peptide, polypeptide, nucleic acid, vector, cell, or composition thereof.

In one aspect, the inventive peptide or polypeptide (protein) can be conjugated to helper peptides or to large carrier molecules to enhance the immunogenicity of the peptide or polypeptide. These molecules include, but are not limited to, influenza peptide, tetanus toxoid, tetanus toxoid CD4 epitope, Pseudomonas exotoxin A, poly-L-lysine, a lipid tail, endoplasmic reticulum (ER) signal sequence, and the like.

The inventive peptide or polypeptide (protein) also can be conjugated to an immunoglobulin molecule using art-accepted methods. The immunoglobulin molecule can be specific for a surface receptor present on tumor cells, but absent or in very low amounts on normal cells. The immunoglobulin also can be specific for a specific tissue (e.g., breast, ovarian, colon, or prostate tissue). Such a peptide-immunoglobulin conjugate or polypeptide-immunoglobulin conjugate allows for targeting of the peptide to a specific tissue and/or cell.

The peptide, polypeptide, nucleic acid, vector, cell, or composition thereof is administered to a host (e.g., mammal, such as a human) in an amount effective to generate an immune response, preferably a cellular immune response. The efficacy of the peptide, polypeptide, nucleic acid, vector, or cell as an immunogen may be determined by in vivo or in vitro parameters as are known in the art. These parameters include but are not limited to antigen-specific cytotoxicity assays, regression of tumors, inhibition of cancer cells, production of cytokines, and the like.

Any suitable dose of the peptide, polypeptide, nucleic acid, vector, or cell or composition thereof can be administered to a host. The appropriate dose will vary depending upon such factors as the host's age, weight, height, sex, general medical condition, previous medical history, disease progression, and tumor burden and can be determined by a clinician. For example, the peptide can be administered in a dose of about 0.05 mg to about 10 mg (e.g., 0.1 mg, 0.5 mg, 1 mg, 2 mg, 3 mg, 4 mg, 5 mg, 6 mg, 7 mg, 8 mg, 9 mg, and ranges therebetween) per vaccination of the host (e.g., mammal, such as a human), and preferably about 0.1 mg to about 5 mg per vaccination. Several doses (e.g., 1, 2, 3, 4, 5, 6, or more) can be provided (e.g., over a period of weeks or months). In one embodiment a dose is provided every month for 3 months.

When the vector is a viral vector, a suitable dose can include about 1×10⁵ to about 1×10¹² (e.g. 1×10⁶, 1×10⁷, 1×10⁸, 1×10⁹, 1×10¹⁰, 1×10¹¹, and ranges g, therebetween) plaque forming units (pfus), although a lower or higher dose can be administered to a host. For example, about 2×10⁸ pfus can be administered (e.g., in a volume of about 0.5 mL).

The inventive cells (e.g., cytotoxic T cells) can be administered to a host in a dose of between about 1×10⁵ and 2×10¹¹ (e.g. 1×10⁶, 1×10⁷, 1×10⁸, 1×10⁹, 1×10¹⁰, and g, ranges therebetween) cells per infusion. The cells can be administered in, for example, one to three (e.g., one, two, or three) infusions. In addition to the administration of the cells, the host can be administered a biological response modifier, such as interleukin 2 (IL-2). When the cells to be administered are cytotoxic T cells, the administration of the cytotoxic T cells can be followed by the administration of the peptide, polypeptide, nucleic acid, vector, or composition thereof in order to prime the cytotoxic T cells to further expand the T cell number in vivo.

When the cells to be administered are dendritic cells, the amount of dendritic cells administered to the subject will vary depending on the condition of the subject and should be determined via consideration of all appropriate factors by the practitioner. Preferably, about 1×10⁶ to about 1×10¹² (e.g., about 1×10⁷, about 1×10⁸, about 1×10⁹, about 1×10¹⁰, or about 1×10¹¹ including ranges between of any of the cell numbers described herein) dendritic cells are utilized for adult humans. These amounts will vary depending on the age, weight, size, condition, sex of the subject, the type of tumor to be treated, the route of administration, whether the treatment is regional or systemic, and other factors. Those skilled in the art should be readily able to derive appropriate dosages and schedules of administration to suit the specific circumstance and needs of the subject.

The invention provides a method of generating peptide-specific cytotoxic T lymphocytes in vivo, ex vivo, or in vitro by stimulation of lymphocytes with an effective amount of the inventive peptide, polypeptide, nucleic acid, vector, or cell, alone or in a composition with one or more immunostimulatory/regulatory molecules and/or adjuvants or in a liposome formulation. The lymphocytes can be lymphocytes from any suitable source, e.g., peripheral blood, tumor tissues, lymph nodes, and effusions, such as pleural fluid or ascites fluid.

The HERVs peptide specific cytotoxic T lymphocytes are immunoreactive. Preferably, the cytotoxic T lymphocytes inhibit the occurrence of tumor cells and cancer and inhibit the growth of, or kill, tumor cells. The cytotoxic T lymphocytes, in addition to being antigen specific, can be MHC class (e.g., MHC class I) restricted. The cytotoxic T lymphocytes preferably have a CD8⁺ phenotype.

In one embodiment, lymphocytes are removed from the host and stimulated ex vivo with the peptide, polypeptide, nucleic acid, vector, cell, or composition thereof to generate cytotoxic T lymphocytes. The cytotoxic T lymphocytes can be administered to the host in order to enhance an immune response to cancer, thereby inhibiting the cancer. Accordingly, the invention provides a method of inhibiting cancer in a host comprising (a) obtaining lymphocytes (e.g., from the host), (b) stimulating the lymphocytes with the peptide, polypeptide, nucleic acid, vector, cell, or composition thereof to generate cytotoxic T lymphocytes, and (c) administering the cytotoxic T lymphocytes to the host, wherein the cancer is inhibited.

In another embodiment, lymphocytes within the host are stimulated by administration to the host of the peptide, polypeptide, nucleic acid, vector, cell, or composition thereof to generate cytotoxic T lymphocytes, which cytotoxic T lymphocytes enhance an immune response to cancer, thereby inhibiting the cancer.

The invention includes a prime and boost protocol. In particular, in one embodiment related to peptides, polypeptides, and vectors of the invention, the protocol includes an initial “prime” with a composition comprising one or more recombinant vectors encoding the inventive peptide or polypeptide and optionally one or more immunostimulatory/regulatory molecules and/or other tumor-associated antigens (e.g., CEA, MUC1, PSA, and/or Brachyury), modified versions thereof, and immunogenic epitopes thereof, followed by one or preferably multiple “boosts” with a composition containing the inventive peptide or polypeptide or one or more poxvirus vectors encoding the inventive peptide or polypeptide and optionally one or more immunostimulatory/regulatory molecules and/or other tumor-associated antigens (e.g., CEA, MUC1, PSA, and/or Brachyury), modified versions thereof, and immunogenic epitopes thereof.

In this embodiment, the initial priming vaccination can comprise one or more vectors. In one embodiment, a single vector (e.g., poxvirus vector) is used for delivery of the inventive peptide and one or more immunostimulatory/regulatory molecules and/or other tumor-associated antigens (e.g., CEA, MUC1, PSA, and/or Brachyury), modified versions thereof, and immunogenic epitopes thereof. In another embodiment, two or more vectors (e.g., poxvirus vectors) comprise the priming vaccination, which are administered simultaneously in a single injection.

The boosting vaccinations also can comprise one or more vectors (e.g., poxvirus vectors). In one embodiment, a single vector is used for delivery of the inventive peptide and the one or more immunostimulatory/regulatory molecules and/or other tumor-associated antigens (e.g., CEA, MUC1, PSA, and/or Brachyury), modified versions thereof, and immunogenic epitopes thereof of the boosting vaccination. In another embodiment, two or more vectors comprise the boosting vaccination, which are administered simultaneously in a single injection.

Different vectors (e.g., poxvirus vectors) can be used to provide a heterologous prime/boost protocol using vectors carrying different sets of therapeutic molecules for inoculations at different time intervals. For example, in one heterologous prime/boost combination, a first orthopox vector composition is used to prime, and a second avipox vector composition is used to boost.

The schedule for administration of the vectors (e.g., poxvirus vectors) typically involves repeated administration of the boosting vector. The boosting vector can be administered 1-3 times (e.g., 1, 2, or 3 times) at any suitable time period (e.g., every 2-4 weeks) for any suitable length of time (e.g., 6-12 weeks for a total of at least 5 to 15 boosting vaccinations). For example, the primary vaccination can comprise a recombinant vaccinia or MVA vector followed by multiple booster vaccinations with an avipox vector. In a particular embodiment, the host receives one vaccination with the priming vector, followed every 2 weeks thereafter with the boosting vector for 6 boosts, followed by every 4 weeks thereafter with the boosting vector, and continuing with the boosting vector for a period of time dependent on disease progression.

The invention further provides a kit that, in one embodiment, has at least a first recombinant vector (e.g., poxvirus vector) that has incorporated into its genome or portion thereof a nucleic acid encoding the inventive peptide or polypeptide in a pharmaceutically acceptable carrier. The first recombinant vector (e.g., poxvirus vectors) also can comprise one or more nucleic acids encoding one or more immunostimulatory/regulatory molecules and/or other tumor-associated antigens (e.g., CEA, MUC1, PSA, and/or Brachyury), modified versions thereof, and immunogenic epitopes thereof. In addition to the first recombinant vector, the kit can have a second recombinant vector that comprises one or more nucleic acids encoding one or more immunostimulatory/regulatory molecules and/or other tumor-associated antigens (e.g., CEA, MUC1, PSA, and/or Brachyury), modified versions thereof, and immunogenic epitopes thereof in a pharmaceutically acceptable carrier. The kit further provides containers, injection needles, and instructions on how to use the kit. In another embodiment, the kit further provides an adjuvant such as GM-CSF and/or instructions for use of a commercially available adjuvant with the kit components.

Accordingly, the invention provides a method of inducing an immune response against cancer in a subject comprising (a) administering to the subject a first vector (e.g., viral vector, such as a poxviral vector) comprising a nucleic acid encoding the amino acid sequence of any one of SEQ ID NOs: 1-100 and 103 and (b) administering to the subject a second vector (e.g., viral vector, such as a poxviral vector) comprising a nucleic acid encoding the amino acid sequence of any one of SEQ ID NOs: 1-100 and 103.

As discussed above, the peptide, polypeptide, nucleic acid, vector, cell, or composition thereof can be administered to a host by various routes including, but not limited to, subcutaneous, intramuscular, intradermal, intraperitoneal, intravenous, and intratumoral. When multiple administrations are given, the administrations can be at one or more sites in a host and a single dose can be administered by dividing the single dose into equal portions for administration at one, two, three, four or more sites on the individual.

Administration of the peptide, polypeptide, nucleic acid, vector, cell, or composition thereof can be “prophylactic” or “therapeutic.” When provided prophylactically, the peptide, polypeptide, nucleic acid, vector, cell, or composition thereof is provided in advance of tumor formation, or the detection of the development of tumors, with the goal of preventing, inhibiting or delaying the development of tumors; and/or preventing, inhibiting or delaying metastases of tumors and/or generally preventing or inhibiting progression of cancer in an individual, and generally to allow or improve the ability of the host's immune system to fight against a tumor that the host is susceptible of developing. The prophylactic administration of the peptide, polypeptide, nucleic acid, vector, cell, or composition thereof prevents, ameliorates, or delays the cancer. When provided therapeutically, the peptide, polypeptide, nucleic acid, vector, cell, or composition thereof is provided at or after the diagnosis of the cancer, with the goal of ameliorating the cancer, such as by reducing tumor burden in the individual; inhibiting tumor growth in the individual; increasing survival of the individual; and/or preventing, inhibiting, reversing or delaying progression of the cancer in the individual.

When the host has already been diagnosed with cancer (e.g., metastatic cancer), the peptide, polypeptide, nucleic acid, vector, cell, or composition thereof can be administered in conjunction with other therapeutic treatments such as chemotherapy, surgical resection of a tumor, treatment with targeted cancer therapy, allogeneic or autologous stem cell transplantation, T cell adoptive transfer, other immunotherapies, and/or radiation.

In one aspect, the administration of the peptide, polypeptide, nucleic acid, vector, cell, or composition thereof to a host results in a host cell expressing the inventive peptide and optionally one or more immunostimulatory/regulatory molecules and/or other tumor-associated antigens (e.g., CEA, MUC1, PSA, and/or Brachyury), modified versions thereof, and immunogenic epitopes thereof that were co-administered. The inventive peptide can be expressed at the cell surface of the infected host cell. The one or more immunostimulatory/regulatory molecules and/or other tumor-associated antigens (e.g., CEA, MUC1, PSA, and/or Brachyury), modified versions thereof, and immunogenic epitopes thereof can be expressed at the cell surface or may be actively secreted by the host cell. The expression of both the peptide and the immunostimulatory/regulatory molecule provides the necessary MHC restricted peptide to specific T cells and the appropriate signal to the T cells to aid in antigen recognition and proliferation or clonal expansion of antigen specific T cells. The overall result is an upregulation of the immune system. Preferably, the upregulation of the immune response is an increase in antigen specific T-helper lymphocytes and/or cytotoxic lymphocytes, which are able to kill or inhibit the growth of a cancer cell.

There are a variety of suitable formulations of the pharmaceutical composition for the inventive methods. The following formulations for parenteral, subcutaneous, intravenous, intramuscular, and intraperitoneal administration are exemplary and are in no way limiting. One skilled in the art will appreciate that these routes of administering the peptide, polypeptide, nucleic acid, vector, cell, or composition of the invention are known, and, although more than one route can be used to administer a particular compound, a particular route can provide a more immediate and more effective response than another route.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The peptide, polypeptide, nucleic acid, vector, cell, or composition thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethylene glycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, and synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The following examples further illustrate the invention but, of course, should not be construed as in any way limiting its scope.

### Example 1

This example demonstrates the expression of HHLA2 and HEMO env in normal and cancerous tissues.

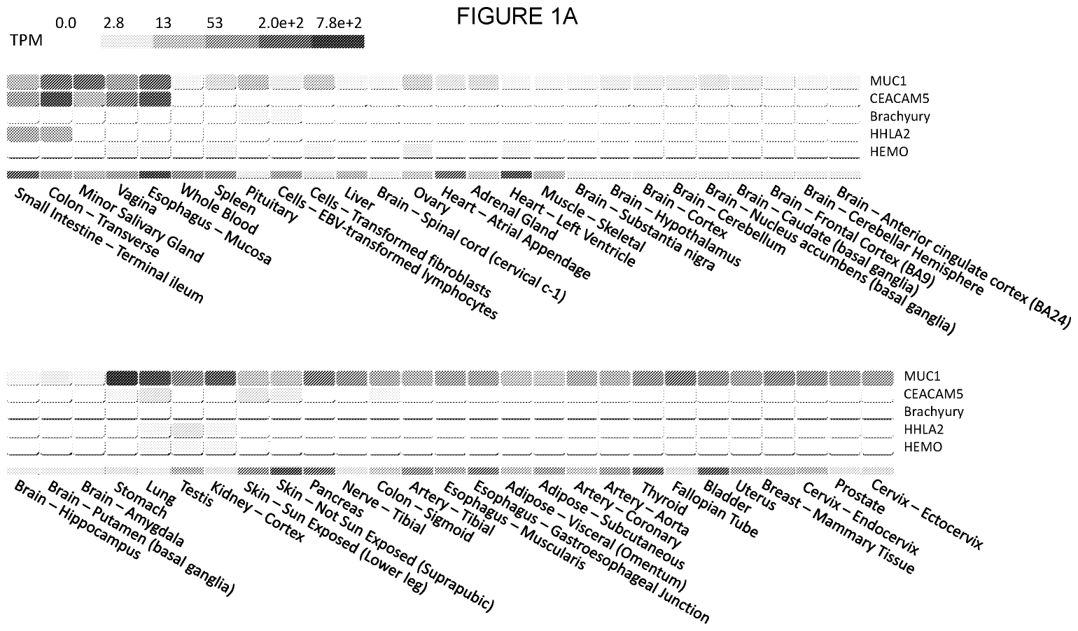

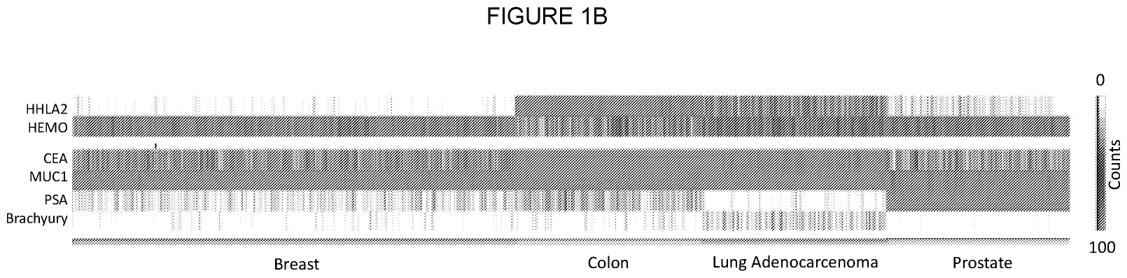

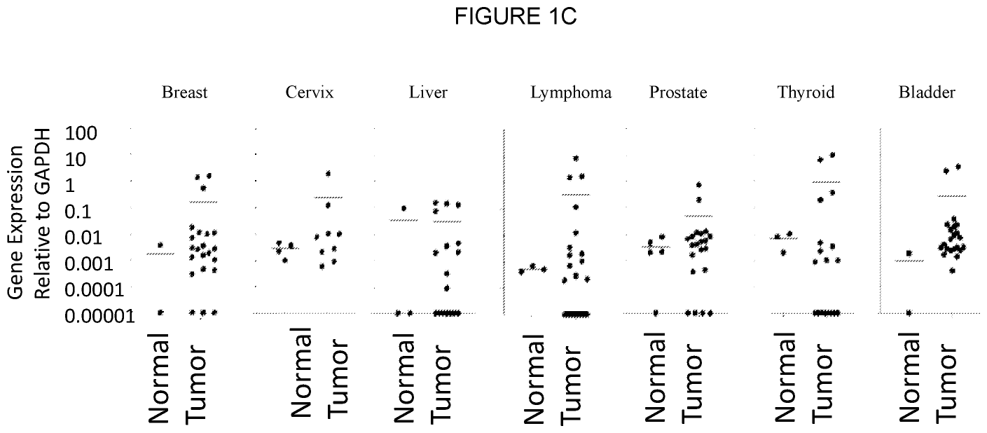

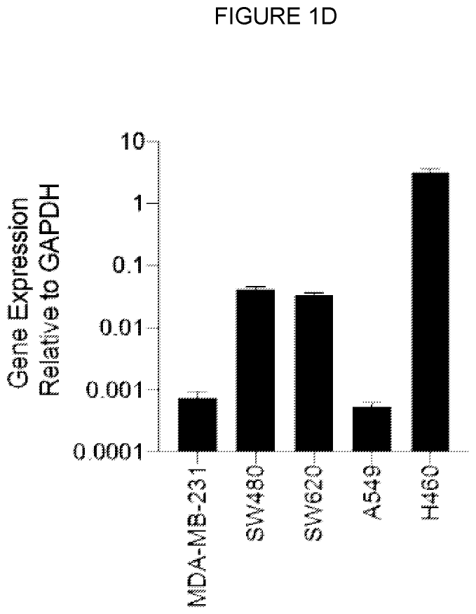

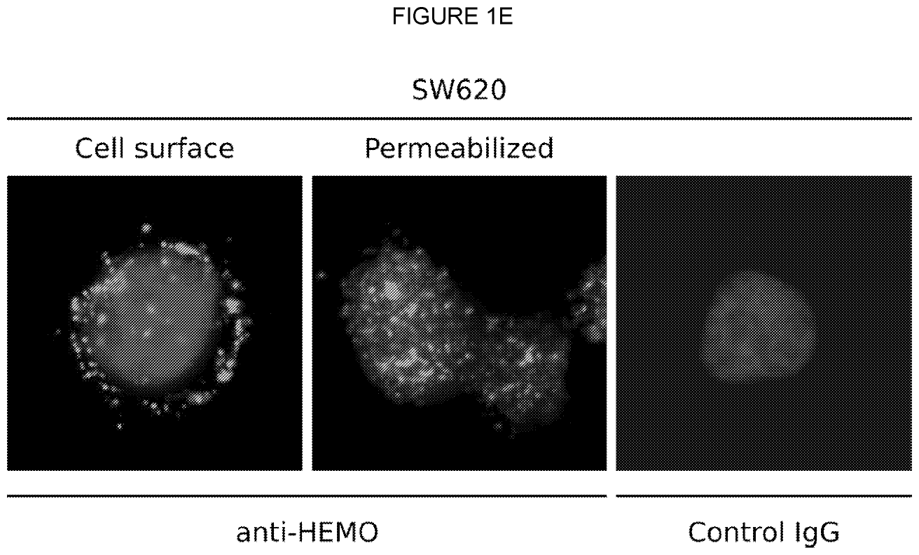

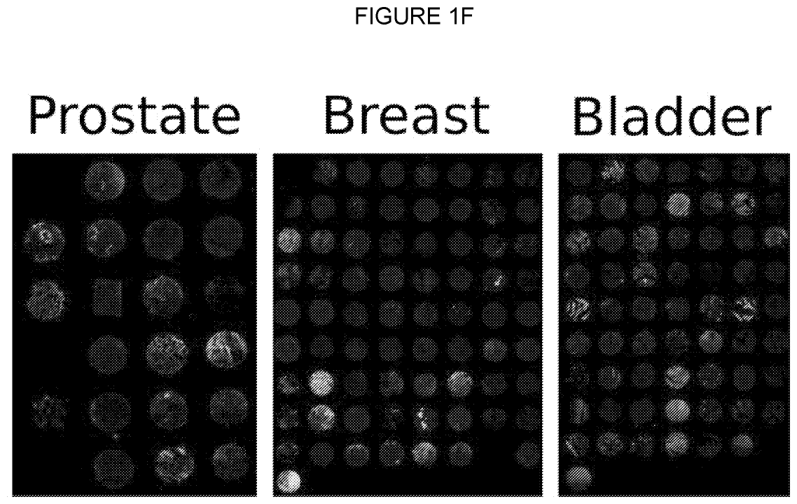

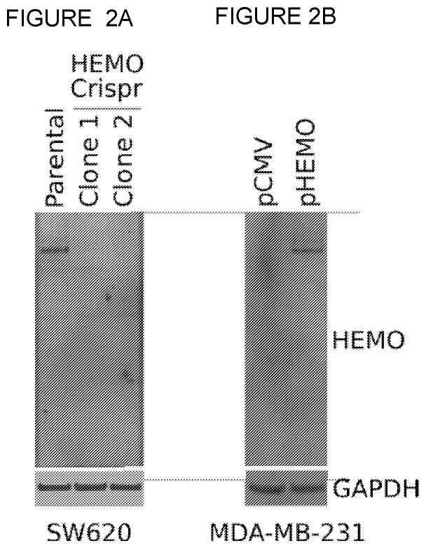

Using in-silico analysis of data in publicly available databases, the expression of HHLA2 and HEMO env transcripts in a panel of normal human tissues (FIG. 1A) and TCGA datasets of human cancers (FIG. 1B) was evaluated. Real-time PCR analysis of relative HEMO mRNA expression levels was also performed in commercially available cDNA libraries from a number of different cancer types and tumor-adjacent ‘normal’ tissues (FIG. 1C). HEMO mRNA expression was detected in multiple human carcinoma cell lines (FIG. 1D).

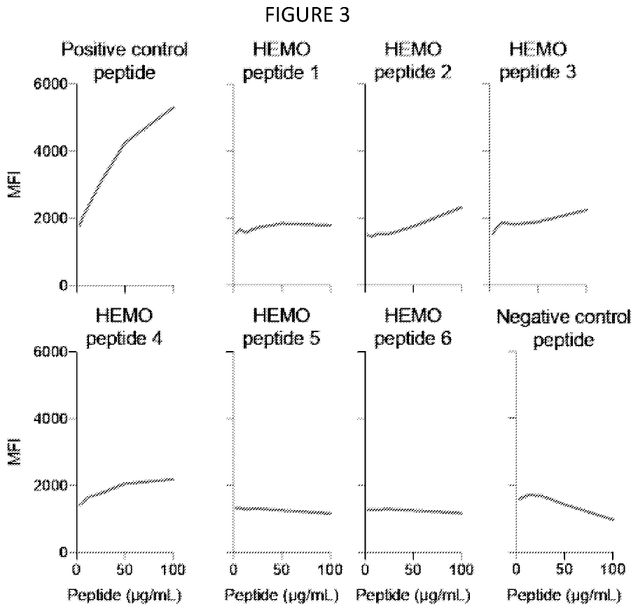

Using a commercially available rabbit HEMO polyclonal antibody, western blot analyses were performed using protein lysates generated from both the parental SW620 cell line and two SW620 clonally-derived cell lines in which HEMO expression was silenced using a CRISPR-based strategy (FIG. 2A).

Additionally, HEMO protein analysis in human breast carcinoma MDA-MB-231 cells expressing either a control plasmid or one encoding a full-length HEMO was examined (FIG. 2B).

These results demonstrated the specificity of the HEMO polyclonal antibody. Using this HEMO polyclonal antibody, an immuno-fluorescent analysis was performed, and it was demonstrated that the HEMO protein was expressed both on the cell surface, and in the cytoplasm of the SW620 cell line (FIG. 1E). Furthermore, HEMO expression in commercially available bladder, breast and prostate tumor tissue micro-arrays was stained for and observed (FIG. 1F).

### Example 2

This example demonstrates the identification of human immunogenic epitopes of HEMO HERVs.

To help define which regions of HEMO will likely be immunogenic, existing algorithms were employed to identify 9-mer native peptides predicted to bind HLA-A2 (Table 1). Based upon these sequences, potential enhancer agonist epitopes were designed (Table 1).

TABLE 1
HEMO 9-mer peptide sequences.

| | | | SEQ |
|---|---|---|---|
| Protein | Designation | Sequence | ID NO: |
| HEMO | | | |
| | HEMO Peptide 1 | RLLEGNFSL | 1 |
| | HEMO Peptide 2 | GLGYLVPSL | 2 |
| | HEMO Peptide 3 | ALLQLTLTA | 3 |
| | HEMO Peptide 4 | LQLTLTAFL | 4 |
| | HEMO Peptide 5 | WMYERVWYP | 5 |
| | HEMO Peptide 6 | WLTGSNLTL | 6 |
| | HEMO Peptide 1 Agonist | RLLEGNFSV | 7 |
| | HEMO Peptide 2 Agonist | GLGYLVPSV | 8 |
| | HEMO Peptide 3 Agonist | ALLQLTLTV | 9 |
| | HEMO Peptide 4 Agonist | LQLTLTAFV | 10 |
| | HEMO Peptide 5 Agonist | WMYERVWYV | 11 |
| | HEMO Peptide 6 Agonist | WLTGSNLTV | 12 |

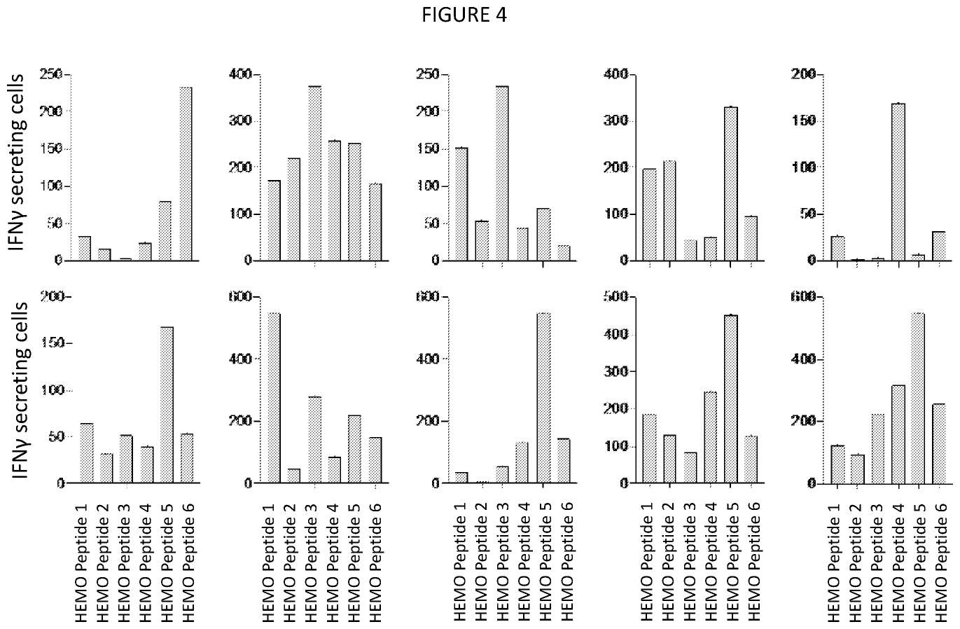

Studies were then conducted to determine binding of these native peptides to human HLA-A2 using an in-vitro binding assay (FIG. 3). As seen in FIG. 3, limited binding was observed. To increase the potential that peptides would be capable of binding to multiple HLA alleles, peptides 15-21 amino acids in length which incorporated these predicted HLA-A2 9-mer peptides, including potential enhancer agonist epitopes, were prepared (Table 2). Using a set of these identified HEMO-derived peptides 20-21 amino-acids in length, a binding prediction algorithm was performed for multiple HLA alleles. Although there continues to be an HLA-A2 bias of these 20-21-mer peptides, several peptides were predicted to bind additional HLA alleles.

TABLE 2
HEMO long peptide sequences.

| Protein | Designation | Sequence | SEQ ID NO: |
|---|---|---|---|
| HEMO | | | |
| | HEMO 15-mer Peptide | LSNYALLQLTLTAFL | 13 |
| | 1 | | |
| | HEMO 15-mer Peptide | WTYSGQWMYERVWYP | 14 |
| | 2 | | |
| | HEMO 15-mer Peptide | WMYERVWYPQAEVQN | 15 |
| | 3 | | |
| | HEMO 15-mer Peptide | SFAQVRLLEGNFSLC | 16 |
| | 4 | | |
| | HEMO 15-mer Peptide | RTPTWWLTGSNLTLS | 103 |
| | 5 | | |
| | HEMO 15-mer Peptide | PTWWLTGSNLTLSVN | 17 |
| | 6 | | |
| | HEMO 15-mer Peptide | KWSGRCGLGYLVPSL | 18 |
| | 7 | | |
| | HEMO 15-mer Peptide | RCGLGYLVPSLTRYL | 19 |
| | 8 | | |
| | HEMO 15-mer agonist | LSNYALLQLTLTVFL | 20 |
| | Peptide 1 | | |
| | HEMO 15-mer agonist | WTYSGQWMYERVWYV | 21 |
| | Peptide 2 | | |
| | HEMO 15-mer agonist | WMYERVWYVQAEVQN | 22 |
| | Peptide 3 | | |
| | HEMO 15-mer agonist | SFAQVRLLEGNFSVC | 23 |
| | Peptide 4 | | |
| | HEMO 15-mer agonist | RTPTWWLTGSNLTVS | 24 |
| | Peptide 5 | | |
| | HEMO 15-mer agonist | PTWWLTGSNLTVSVN | 25 |
| | Peptide 6 | | |
| | HEMO 15-mer agonist | KWSGRCGLGYLVPSV | 26 |
| | Peptide 7 | | |
| | HEMO 15-mer agonist | RCGLGYLVPSVTRYL | 27 |
| | Peptide 8 | | |
| | HEMO 15-mer agonist | LSNYALLQLTLTAFV | 28 |
| | Peptide 9 | | |
| | HEMO 21-mer peptide | GSLSNYALLQLTLTAFLTILV | 29 |
| | 1 | | |
| | HEMO 20-mer peptide | WTYSGQWMYERVWYPQAEVQ | 30 |
| | 2 | | |
| | HEMO 20-mer peptide | LSFAQVRLLEGNFSLCVENK | 31 |
| | 3 | | |
| | HEMO 20-mer peptide | HRTPTWWLTGSNLTLSVNNS | 32 |
| | 4 | | |
| | HEMO 20-mer peptide | KWSGRCGLGYLVPSLTRYLT | 33 |
| | 5 | | |
| | HEMO 21-mer peptide | GSLSNYALLQLTLTAFVTILV | 34 |
| | agonist 1 | | |
| | HEMO 20-mer peptide | WTYSGQWMYERVWYVQAEVQ | 35 |
| | agonist 2 | | |
| | HEMO 20-mer peptide | LSFAQVRLLEGNFSVCVENK | 36 |
| | agonist 3 | | |
| | HEMO 20-mer peptide | HRTPTWWLTGSNLTVSVNNS | 37 |
| | agonist 4 | | |
| | HEMO 20-mer peptide | KWSGRCGLGYLVPSVTRYLT | 38 |
| | agonist 5 | | |
| | HEMO 20-mer peptide | GSLSNYALLQLTLTVFLTILV | 39 |
| | agonist 6 | | |

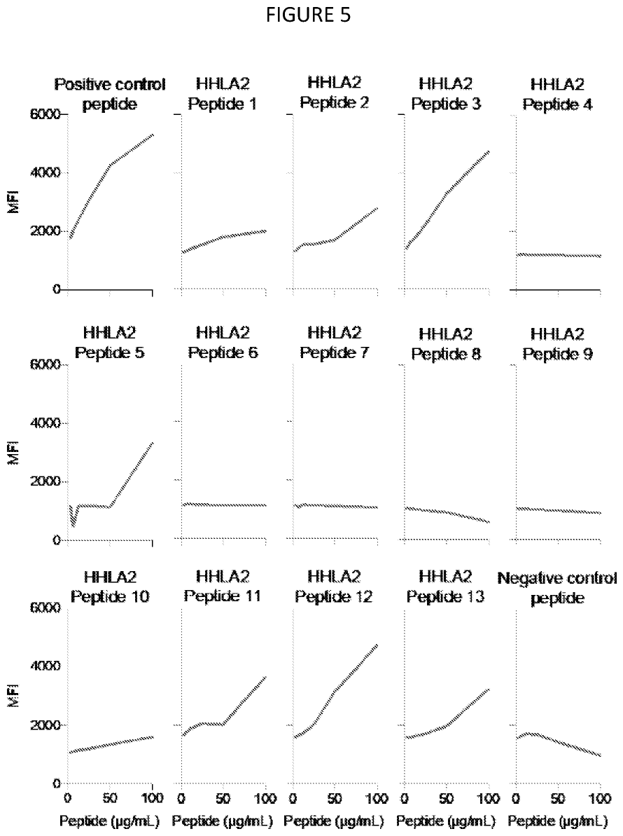

To assess the potential immunogenicity of the HEMO peptides, a limiting dilution analysis of T cell reactivity was performed to each of the native HEMO 9-mer peptides using blood collected from a prostate cancer patient. Briefly, CD8+ T cells isolated form human PBMCs were plated at 1000-5000 cells/well and underwent rapid expansion, followed by an assay for reactive T cells via an IFNγELISPOT assay. The presence of HEMO-reactive T cells specific to a number of these identified epitopes was observed (FIG. 4).

The native and agonist peptides (9-mer and 21 and 20-mer) of HEMO selected for further studies were those with no prior information in the literature as to their potential immunogenicity in humans.

### Example 3

This example demonstrates the characterization of human immunogenic epitopes of HEMO HERVs.

Experiments were conducted to determine if cancer patients who had received prior vaccinations with non-HERV vaccines (vaccines directed against CEA and/or MUC1) exhibited T-cell responses to a pool of native 9-mer HEMO peptides. These were colorectal, appendiceal, and bladder cancer patients (Table 3).

TABLE 3
Description of cancer patients tested for HEMO-
reactive T cells following immunotherapy.

| Patient # | TRIAL | Cancer Type | HLA |
|---|---|---|---|
| 1 | CV301 | appendiceal | HLA A 02, 02 |
| 2 | Ad CEA Avelumab + Folfox | colon | HLA A 02, 03 |
| 3 | BCG +/− PANVAC | bladder | HLA A 02, 68 |

TABLE 4
Description of HLA-A2+ cancer patients tested
for HEMO-reactive T cells following immunotherapy

| Patient # | TRIAL | Cancer Type |
|---|---|---|
| 4 | PANVAC | Colon |
| 5 | PANVAC | Colon |
| 6 | PANVAC | Colon |
| 7 | PANVAC | Colon |
| 8 | PANVAC | Colon |
| 9 | PANVAC | Colon |
| 10 | Avelumab | Colon |
| 11 | Avelumab | Colon |

Patients were shown to possess T-cell responses to HEMO (Table 5).

TABLE 5
T-cell responses to HEMO env 9-mer pool after immunotherapy.

| | | HEMO native 9-mer pool | | | | | | | |
|---|---|---|---|---|---|---|---|---|---|
| | | CD107a + | IFNg + | IL-2 + | TNF + | CD107a + | IFNg + | IL-2 + | TNF + |
| Patient # | Day | CD4 | CD4 | CD4 | CD4 | CD8 | CD8 | CD8 | CD8 |
| 1 | 70 | 116 | 0 | 0 | 0 | 0 | 214 | 71 | 0 |
| | 350 | 0 | 137 | 52 | 687 | 42 | 494 | 46 | 3458 |
| 2 | 118 | 0 | 416 | 0 | 0 | 0 | 0 | 5 | 0 |
| 3 | 106 | 561 | 507 | 134 | 880 | 4682 | 2662 | 54 | 6415 |

TABLE 6
T cell responses to a pool of 9-mer native or
agonist HEMO peptides following immunotherapy

| | | HEMO native or agonist 9-mer pool | | | | | | | |
|---|---|---|---|---|---|---|---|---|---|
| | | CD107a + | IFNg + | IL-2 + | TNF + | CD107a + | IFNg + | IL-2 + | TNF + |
| Patient # | Day | CD4 | CD4 | CD4 | CD4 | CD8 | CD8 | CD8 | CD8 |
| 4 | D 69 | 850 | 0 | 0 | 0 | 0 | 0 | 0 | 0 |
| 5 | D 97 | 0 | 0 | 0 | 170 | 934 | 9 | 0 | 938 |
| 6 | D 69 | 217 | 58 | 73 | 18 | 649 | 15 | 14 | 192 |
| 7 | D 69 | 60 | 0 | 0 | 146 | 0 | 0 | 0 | 0 |
| 8 | D 70 | 620 | 680 | 394 | 1555 | 1159 | 135 | 10 | 767 |
| 9 | D 71 | 0 | 0 | 0 | 0 | 0 | 0 | 29 | 0 |
| 10 | D 99 | 0 | 2359 | 2116 | 2369 | 0 | 0 | 0 | 0 |
| 11 | D 85 | 102 | 0 | 1682 | 73 | 0 | 0 | 0 | 0 |

PBMCs obtained from cancer patients following immunotherapy were stimulated with a pool of six 9-mer native or agonist HEMO Peptides (each native or agonist peptide with better binding included) (Table 1), and T cells were assessed for the production of cytokine or positivity for the degranulation marker CD107a. Values in Tables 5 and 6 indicate the absolute number of CD4+ or CD8+ T cells producing cytokine or positive for CD107a per 1×10⁶ PBMCs. Background was subtracted. Values were considered a response if >250 and >2 fold-change vs the negative control.

Many of those T cells were shown to be multifunctional, expressing more than one Type I cytokine and/or CD107a (Tables 7 and 8), suggestive of a more lytic function.

TABLE 7
Multifunctional T cell responses to a pool of 9-
mer native HEMO peptides following immunotherapy.

| | | | | HEMO native 9-mer pool | |
|---|---|---|---|---|---|
| Patient | | | | CD4 | CD8 |
| # | Trial | Cancer type | Day | 2 or more | 2 or more |
| 1 | CV301 | appendiceal | 70 | 0 | 0 |
| | | | 350 | 385 | 520 |
| 2 | Ad CEA | colon | 118 | 0 | 0 |
| | Avelumab + | | | | |
| | Folfox | | | | |
| 3 | BCG +/− | bladder | 106 | 381 | 4112 |
| | PANVAC | | | | |

TABLE 8
Multifunctional T cell responses to a pool of 9-mer native
or agonist HEMO peptides following immunotherapy.

HEMO native or agonist 9-mer pool

| Patient # | Day | CD107a + CD4 | IFNg + CD4 |
|---|---|---|---|
| 4 | D 69 | 28 | 0 |
| 5 | D 97 | 98 | 832 |
| 6 | D 69 | 16 | 216 |
| 7 | D 69 | 147 | 0 |
| 8 | D 70 | 638 | 504 |
| 9 | D 71 | 0 | 0 |
| 10 | D 99 | 2541 | 0 |
| 11 | D 85 | 1073 | 0 |

PBMCs obtained from cancer patients following immunotherapy were stimulated with a pool of six 9-mer native or agonist HEMO Peptides (each native or agonist peptide with better binding included) (Table 1), and T cells were assessed for the production of cytokine or positivity for the degranulation marker CD107a. Values in Tables 7 and 8 indicate the absolute number of T cells positive for 2 or more markers (IFNg, IL-2, TNF, CD107a) per 1×10⁶ PBMCs. Background was subtracted. Values were considered a response if >250 and >2 fold-change vs the negative control.

Studies additionally employed PBMCs from a patient pre- vs. post-treatment with a non-HERV—based vaccine. In that study, the patient was shown to have an ongoing T-cell response to HEMO and also to mount specific T-cell responses to the selected HEMO peptides post- vs. pre-treatment (Tables 9 and 10), wherein some of those T-cells were shown to be multifunctional (Tables 11 and 12).

TABLE 9
T cell responses to a pool of 9-mer native HEMO peptides before and after immunotherapy.

| | | HEMO native 9-mer pool | | | | | | | |
|---|---|---|---|---|---|---|---|---|---|
| | | CD107a + | IFNg + | IL-2 + | TNF + | CD107a + | IFNg + | IL-2 + | TNF + |
| Patient # | Day | CD4 | CD4 | CD4 | CD4 | CD8 | CD8 | CD8 | CD8 |
| 1 | 0 | 0 | 0 | 464 | 321 | 988 | 62 | 4 | 637 |
| | 70 | 116 | 0 | 0 | 0 | 0 | 214 | 71 | 0 |
| | 350 | 0 | 137 | 52 | 687 | 42 | 494 | 46 | 3458 |

TABLE 10
T cell responses to a pool of 9-mer native or agonist
HEMO peptides before and after immunotherapy.

| | | HEMO native or agonist 9-mer pool | | | | | | | |
|---|---|---|---|---|---|---|---|---|---|
| | | CD107a + | IFNg + | IL-2 + | TNF + | CD107g + | IFNg + | IL-2 + | TNF + |
| Patient # | ^{I}Day | CD4 | CD4 | CD4 | CD4 | CD8 | CD8 | CD8 | CD8 |
| 4 | Pre | 1744 | 71 | 70 | 2066 | 2071 | 0 | 21 | 0 |
| | D 69 | 850 | 0 | 0 | 0 | 0 | 0 | 0 | 0 |
| 6 | Pre | 117 | 46 | 414 | 231 | 23 | 0 | 14 | 200 |
| | D 69 | 217 | 58 | 73 | 18 | 649 | 15 | 14 | 192 |
| 7 | Pre | 0 | 0 | 0 | 0 | 0 | 0 | 9 | 0 |
| | D 69 | 60 | 0 | 0 | 146 | 0 | 0 | 0 | 0 |
| 8 | Pre | 0 | 68 | 32 | 76 | 0 | 128 | 15 | 6 |
| | D 70 | 620 | 680 | 394 | 1555 | 1159 | 135 | 10 | 767 |
| 9 | Pre | 104 | 0 | 0 | 641 | 0 | 0 | 16 | 0 |
| | D 71 | 0 | 0 | 0 | 0 | 0 | 0 | 29 | 0 |
| 11 | Pre | 21 | 15 | 1 | 0 | 332 | 0 | 0 | 856 |
| | D 85 | 102 | 0 | 1682 | 73 | 0 | 0 | 0 | 0 |

PBMCs obtained from a cancer patient before and after immunotherapy were stimulated with a pool of six 9-mer native or agonist HEMO peptides (each native or agonist peptide with better binding included) (Table 1), and T cells were assessed for the production of cytokine or positivity for the degranulation marker CD107a. Values in Tables 9 and 10 indicate the absolute number of CD4+ or CD8+ T cells producing cytokine or positive for CD107a per 1×10⁶ PBMCs. Background was subtracted. Values were considered a response if >250 and >2 fold-change vs the negative control.

TABLE 11
Multifunctional T cell responses to a pool of 9-mer
native HEMO peptides before and after immunotherapy.

| | | | | HEMO native 9-mer pool | |
|---|---|---|---|---|---|
| Patient | | | | CD4 | CD8 |
| # | Trial | Cancer type | Day | 2 or more | 2 or more |
| 1 | CV301 | Appendiceal | 1 | 156 | 620 |
| | | | 70 | 0 | 0 |
| | | | 350 | 385 | 520 |

TABLE 12
Multifunctional T cell responses to a pool of 9-mer native
or agonist HEMO peptides before and after immunotherapy.

HEMO native or agonist 9-mer pool

| Patient # | Day | CD4 2 or more | CD8 2 or more |
|---|---|---|---|
| 4 | Pre | 104 | 40 |
| | D 69 | 28 | 0 |
| 6 | Pre | 306 | 171 |
| | D 69 | 16 | 216 |
| 7 | Pre | 0 | 0 |
| | D 69 | 147 | 0 |
| 8 | Pre | 34 | 0 |
| | D 70 | 638 | 504 |
| 9 | Pre | 52 | 0 |
| | D 71 | 0 | 0 |
| 11 | Pre | 0 | 229 |
| | D 85 | 1073 | 0 |

PBMCs obtained from a cancer patient before and after immunotherapy were stimulated with a pool of six 9-mer native or agonist HEMO peptides (each native or agonist peptide with better binding included) (Table 1), and T cells were assessed for the production of cytokine or positivity for the degranulation marker CD107a. Values in Tables 11 and 12 indicate the absolute number of T cells positive for 2 or more markers (IFNg, IL-2, TNF, CD107a) per 1×10⁶ PBMCs. Background was subtracted. Values were considered a response if >250 and >2 fold-change vs the negative control.

These studies indicated that HEMO specific responses were most likely the consequence of tumor cell destruction and subsequent epitope spreading or so-called “antigen cascade.” The studies also further demonstrated the immunogenicity of these specific HEMO peptides in humans, and their potential for use in anti-cancer vaccination.

### Example 4

This example demonstrates the identification of human immunogenic epitopes of HHLA2 HERVs.

To help define regions of HHLA2 HERV that will likely be immunogenic, existing algorithms were employed to identify native 9-mer peptides predicted to bind HLA-A2 (Table 8). Numerous potential enhancer agonist epitopes were designed (Table 8).

TABLE 13
HHLA2 9-mer peptide sequences.

| | | | SEQ |
|---|---|---|---|
| Protein | Designation | Sequence | ID NO: |
| HHLA2 | | | |
| | HHLA2 Peptide 1 | FLICSVLSV | 40 |
| | HHLA2 Peptide 2 | GIFPLAFFI | 41 |
| | HHLA2 Peptide 3 | GLWILVPSA | 42 |
| | HHLA2 Peptide 4 | RMKSGTFSV | 43 |
| | HHLA2 Peptide 5 | YTLLTIHTV | 44 |
| | HHLA2 Peptide 6 | YLSSSQNTI | 45 |
| | HHLA2 Peptide 7 | ALSFFLILI | 46 |
| | HHLA2 Peptide 8 | AAFLLIWSV | 47 |
| | HHLA2 Peptide 9 | SLLDEGIYT | 48 |
| | HHLA2 Peptide 10 | LLDEGIYTC | 49 |
| | HHLA2 Peptide 11 | AQTALSFFL | 50 |
| | HHLA2 Peptide 12 | KVGVFLTPV | 51 |
| | HHLA2 Peptide 13 | LLTIHTVHV | 52 |
| | HHLA2 Peptide 2 Agonist | GIFPLAFFV | 53 |
| | HHLA2 Peptide 3 Agonist | GLWILVPSV | 54 |
| | HHLA2 Peptide 6 Agonist | YLSSSQNTV | 55 |
| | HHLA2 Peptide 7 Agonist | ALSFFLILV | 56 |
| | HHLA2 Peptide 9 Agonist | SLLDEGIYV | 57 |
| | HHLA2 Peptide 10 Agonist | LLDEGIYTV | 58 |
| | HHLA2 Peptide 11 Agonist | AQTALSFFV | 59 |

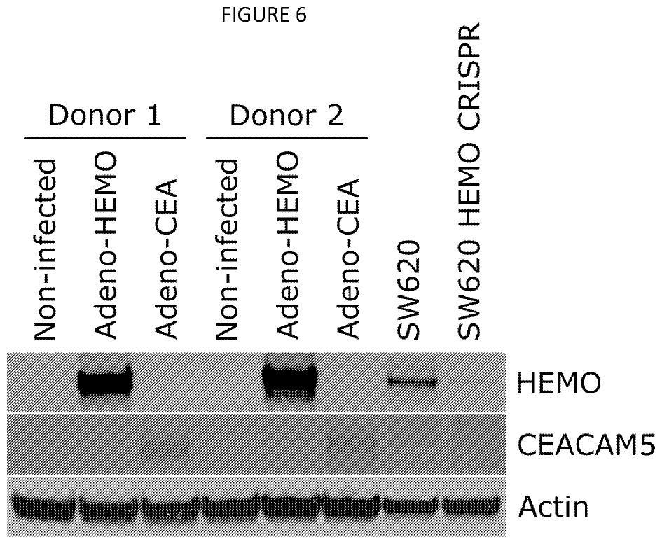

Studies were then conducted to determine binding of these native peptides to human HLA-A2 in an in-vitro binding assay (FIG. 5).

To increase the potential that peptides would be capable of binding to multiple additional HLA alleles, peptides 15-20 amino acids in length which incorporated the HLA-A2 9-mer peptides, including potential enhancer agonist epitopes, were designed (Table 9).

TABLE 14
HHLA2 long peptide sequences.

| Protein | Designation | Sequence | SEQ ID NO: |
|---|---|---|---|
| HHLA2 | | | |
| | HHLA2 15-mer peptide 1 | MKAQTALSFFLILI | 60 |
| | HHLA2 15-mer peptide 2 | QTALSFFLILITSLS | 61 |
| | HHLA2 15-mer peptide 3 | SLSGSQGIFPLAFFI | 62 |
| | HHLA2 15-mer peptide 4 | SQGIFPLAFFIYVPM | 63 |
| | HHLA2 15-mer peptide 5 | LFFRRVSLLDEGIYT | 64 |
| | HHLA2 15-mer peptide 6 | RRVSLLDEGIYTCYV | 65 |
| | HHLA2 15-mer peptide 7 | NKVVLKVGVFLTPVM | 66 |
| | HHLA2 15-mer peptide 8 | VLKVGVFLTPVMKYE | 67 |
| | HHLA2 15-mer peptide 9 | KRNTNSFLICSVLSV | 68 |
| | HHLA2 15-mer peptide 10 | NSFLICSVLSVYPRP | 69 |
| | HHLA2 15-mer peptide 11 | WSRMKSGTFSVLAYY | 70 |
| | HHLA2 15-mer peptide 12 | LAYYLSSSQNTIINE | 71 |
| | HHLA2 15-mer peptide 13 | SDEYTLLTIHTVHVE | 72 |
| | HHLA2 15-mer peptide 14 | TASHNKGLWILVPSA | |
| | HHLA2 15-mer peptide 15 | GLWILVPSAILAAFL | 74 |
| | HHLA2 15-mer peptide 16 | VPSAILAAFLLIWSV | 75 |
| | HHLA2 15-mer peptide 17 | SAILAAFLLIWSVKC | 76 |
| | HHLA2 15-mer agonist | MKAQTALSFFLILV | 77 |
| | peptide 1 | | |
| | HHLA2 15-mer agonist | QTALSFFLIVITSLS | 78 |
| | peptide 2 | | |
| | HHLA2 15-mer agonist | SLSGSQGIFPLAFFV | 79 |
| | peptide 3 | | |
| | HHLA2 15-mer agonist | SQGIFPLAFFVYVPM | 80 |
| | peptide 4 | | |
| | HHLA2 15-mer agonist | LFFRRVSLLDEGIYV | 81 |
| | peptide 5 | | |
| | HHLA2 15-mer agonist | RRVSLLDEGIYVCYV | 82 |
| | peptide 6 | | |
| | HHLA2 15-mer agonist | LAYYLSSSQNTVINE | 83 |
| | peptide 12 | | |
| | HHLA2 15-mer agonist | TASHNKGLWILVPSV | 84 |
| | peptide 14 | | |
| | HHLA2 15-mer agonist | GLWILVPSVILAAFL | 85 |
| | peptide 15 | | |
| | HHLA2 20-mer peptide 1 | MKAQTALSFFLILITSLSGS | 86 |
| | HHLA2 20-mer peptide 2 | SLSGSQGIFPLAFFIYVPMN | 87 |
| | HHLA2 20-mer peptide 3 | LFFRRVSLLDEGIYTCYVGTA | 88 |
| | HHLA2 20-mer peptide 4 | TNKVVLKVGVFLTPVMKYEK | 89 |
| | HHLA2 20-mer peptide 5 | KRNTNSFLICSVLSVYPRPI | 90 |
| | HHLA2 20-mer peptide 6 | FKVTWSRMKSGTFSVLAYYL | 91 |
| | HHLA2 20-mer peptide 7 | FSVLAYYLSSSQNTIINESR | 92 |
| | HHLA2 20-mer peptide 8 | NISSDEYTLLTIHTVHVEPSQE | 93 |
| | HHLA2 20-mer peptide 9 | TASHNKGLWILVPSAILAAF | 94 |
| | HHLA2 20-mer peptide 10 | VPSAILAAFLLIWSVKCCRA | 95 |
| | HHLA2 20-mer peptide | MKAQTALSFFLILVTSLSGS | 96 |
| | agonist 1 | | |
| | HHLA2 20-mer peptide | SLSGSQGIFPLAFFVYVPMN | 97 |
| | agonist 2 | | |
| | HHLA2 20-mer peptide | LFFRRVSLLDEGIYVCYVGTA | 98 |
| | agonist 3 | | |
| | HHLA2 20-mer peptide | FSVLAYYLSSSQNTVINESR | 99 |
| | agonist 7 | | |
| | HHLA2 20-mer peptide | TASHNKGLWILVPSVILAAF | 100 |
| | agonist 9 | | |

Using a set of these identified HHLA2-derived peptides 20 amino-acids in length, a binding prediction algorithm was performed for multiple HLA alleles. Although there continues to be an HLA-A2 bias of these 20-mer peptides, several peptides were predicted to bind other HLA alleles.

The native and agonist peptides (9-mer and 20-mer) of HHLA2 selected for further studies were those with no prior information in the literature as to their potential immunogenicity in humans.

### Example 5

This example demonstrates the characterization of human immunogenic epitopes of HHLA2 HERVs.

Experiments were performed to determine if patients with colorectal, appendiceal, and bladder cancer, who had received prior vaccinations with non-HERV vaccines (vaccines directed against CEA and/or MUC1), exhibited T-cell responses to a pool of native 9-mer HHLA2 peptides (Table 15). Patients were shown to possess T-cell responses to HHLA2 (Table 16), and some of those T cells were shown to be multifunctional, expressing more than 1 Type I cytokine and/or CD107a (Table 17), suggestive of a more lytic function.

Studies additionally employed PBMCs from a patient pre- vs. post-treatment with a non-HERV—based vaccine. In that study, the patient was shown to possess some T-cell response to the HHLA2 peptides, and to mount specific T-cell responses to the selected HHLA2 peptides post- vs. pre-treatment (Table 18). Multifunctional T cells against HHLA2 were only detectable after vaccination (Table 19).

These studies indicated that HHLA2 specific responses were most likely the consequence of tumor cell destruction and subsequent epitope spreading or so-called “antigen cascade.” They also further demonstrated the immunogenicity of these specific HHLA2 peptides in humans, and their potential for use in anti-cancer vaccination.

TABLE 15
Description of cancer patients tested for HHLA2-
reactive T cells following immunotherapy.

| Patient # | TRIAL | Cancer Type | HLA |
|---|---|---|---|
| 1 | CV301 | appendiceal | HLA A 02, 02 |
| 2 | Ad CEA Avelumab + Folfox | colon | HLA A 02, 03 |
| 3 | BCG +/− PANVAC | bladder | HLA A 02, 68 |

TABLE 16
T cell responses to a pool of 9-mer native HHLA2 peptides following immunotherapy.

| | | HHLA2 native 9-mer pool | | | | | | | |
|---|---|---|---|---|---|---|---|---|---|
| | | CD107a + | IFNg + | IL-2 + | TNF + | CD107a + | IFNg + | IL-2 + | TNF + |
| Patient # | Day | CD4 | CD4 | CD4 | CD4 | CD8 | CD8 | CD8 | CD8 |
| 1 | 70 | 301 | 0 | 0 | 0 | 0 | 0 | 81 | 0 |
| | 350 | 0 | 162 | 0 | 857 | 1796 | 2344 | 54 | 10890 |
| 2 | 118 | 0 | 471 | 0 | 85 | 0 | 0 | 110 | 0 |
| 3 | 106 | 587 | 482 | 122 | 424 | 3461 | 1393 | 26 | 3197 |

PBMCs obtained from cancer patients following immunotherapy were stimulated with a pool of thirteen 9-mer native HHLA2 peptides (Table 13), and T cells were assessed for the production of cytokine or positivity for the degranulation marker CD107a. Values in Table 16 indicate the absolute number of CD4+ or CD8+ T cells producing cytokine or positive for CD107a per 1×10⁶ PBMCs. Background was subtracted. Values were considered a response if >250 and >2 fold-change vs the negative control.

TABLE 17
Multifunctional T cell responses to a pool of 9-mer
native HHLA2 peptides following immunotherapy.

| | | | | HHLA2 native 9-mer pool | |
|---|---|---|---|---|---|
| Patient | | | | CD4 | CD8 |
| # | Trial | Cancer type | Day | 2 or more | 2 or more |
| 1 | CV301 | appendiceal | 70 | 0 | 0 |
| | | | 350 | 156 | 2827 |
| 2 | Ad CEA | colon | 118 | 58 | 0 |
| | Avelumab + | | | | |
| | Folfox | | | | |
| 3 | BCG +/− | bladder | 106 | 235 | 1901 |
| | PANVAC | | | | |

PBMCs obtained from cancer patients following immunotherapy were stimulated with a pool of thirteen 9-mer native HHLA2 peptides (Table 13), and T cells were assessed for the production of cytokine or positivity for the degranulation marker CD107a. Values in Table 17 indicate the absolute number of T cells positive for 2 or more markers (IFNg, IL-2, TNF, CD107a) per 1×10⁶ PBMCs. Background was subtracted. Values were considered a response if >250 and >2 fold-change vs the negative control.

TABLE 18
T cell responses to a pool of 9-mer native HHLA2 peptides before and after immunotherapy.

| | | HHLA2 native 9- mer pool | | | | | | | |
|---|---|---|---|---|---|---|---|---|---|
| | | CD107a + | IFNg + | IL-2 + | TNF + | CD107a + | IFNg + | IL-2 + | TNF + |
| Patient # | Day | CD4 | CD4 | CD4 | CD4 | CD8 | CD8 | CD8 | CD8 |
| 1 | 1 | 0 | 0 | 300 | 0 | 562 | 0 | 30 | 0 |
| | 70 | 301 | 0 | 0 | 0 | 0 | 0 | 81 | 0 |
| | 350 | 0 | 162 | 0 | 857 | 1796 | 2344 | 54 | 10890 |

PBMCs obtained from cancer patients following immunotherapy were stimulated with a pool of thirteen 9-mer native HHLA2 peptides (Table 13), and T cells were assessed for the production of cytokine or positivity for the degranulation marker CD107a. Values in Table 18 indicate the absolute number of CD4+ or CD8+ T cells producing cytokine or positive for CD107a per 1×10⁶ PBMCs. Background was subtracted. Values were considered a response if >250 and >2 fold-change vs the negative control.

TABLE 19
Multifunctional T cell responses to a pool of 9-mer
native HHLA2 peptides before and after immunotherapy.

| | | | | HHLA2 native 9-mer pool | |
|---|---|---|---|---|---|
| Patient | | | | CD4 | CD8 |
| # | Trial | Cancer type | Day | 2 or more | 2 or more |
| 1 | CV301 | Appendiceal | 1 | 0 | 41 |
| | | | 70 | 0 | 0 |
| | | | 350 | 156 | 2827 |

PBMCs obtained from cancer patients following immunotherapy were stimulated with a pool of thirteen 9-mer native HHLA2 peptides (Table 13), and T cells were assessed for the production of cytokine or positivity for the degranulation marker CD107a. Values in Table 19 indicate the absolute number of T cells positive for 2 or more markers (IFNg, IL-2, TNF, CD107a) per 1×10⁶ PBMCs. Background was subtracted. Values were considered a response if >250 and >2 fold-change vs the negative control.

### Example 6

This example demonstrates that vaccination with HEMO peptides has an anti-tumor effect.

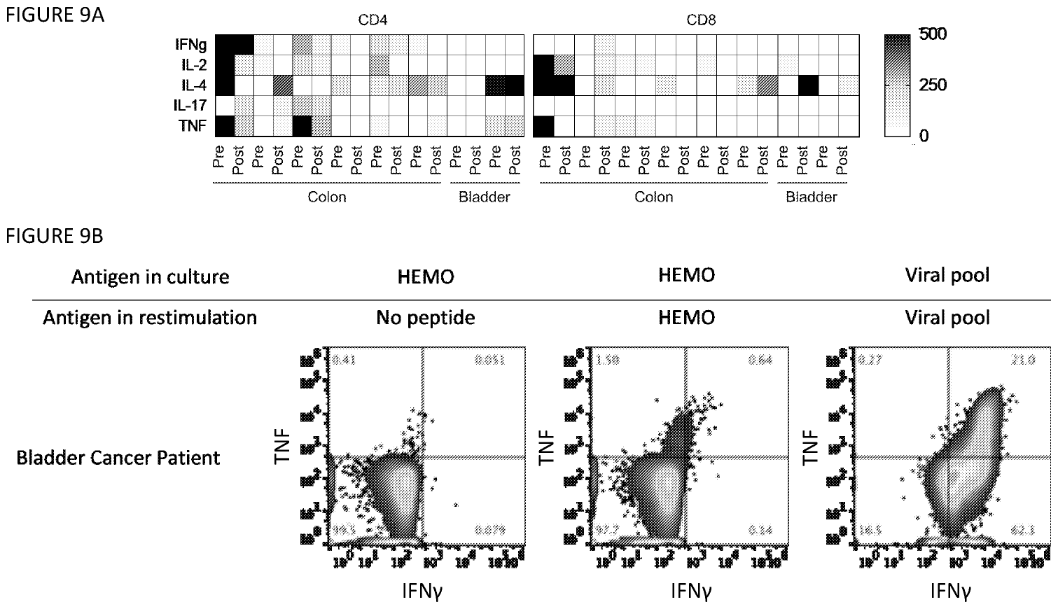

HEMO and other human proteins were compared and a region of homology was identified within the transmembrane region of HEMO (see FIG. 9A). Using areas with no sequence homology, an overlapping peptide library of 15-mers was designed for use in immune assays (se FIG. 9B). A total of 94 15-mer peptides were included in the mix.

PBMCs from colon and bladder cancer patients obtained both pre- and post-immunotherapy were stimulated in-vitro for one week with the 15-mer overlapping HEMO peptide library (94-peptide mix). FIG. 9C is a heatmap representation of the number of antigen-dependent, cytokine-producing CD4+ and CD8+ T cells per 1×10\hat{ }5 CD4+ or CD8+ T cells following an overnight stimulation with the same peptides. FIG. 9D depicts an example of a bladder cancer patient with a robust anti-HEMO immune response following the in-vitro stimulation as described above as assessed by the production of TNF and IFNγ as assessed by flow cytometry.

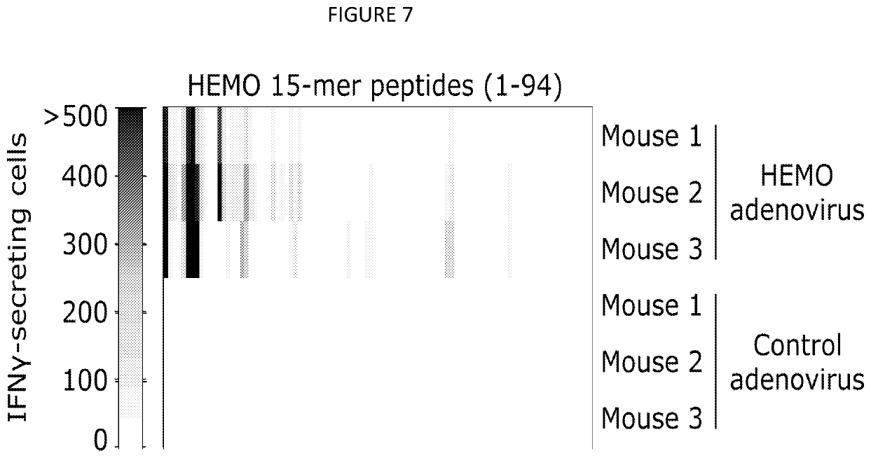

An adenoviral vector encoding the full-length HEMO protein (Adeno-HEMO) was produced, as well as an adenoviral vector encoding human carcinoembryonic antigen (CEA) for use as a control. Human dendritic cells were generated in culture from monocytic cell fractions from peripheral blood mononuclear cells from two normal donors (Donors 1 and 2). Dendritic cells were infected in culture with Adeno-HEMO or control Adeno-CEA. As a positive control, colon carcinoma SW620 cells were used, which express endogenous HEMO protein. As negative controls, non-infected dendritic cells and SW620 cells silenced for HEMO expression via CRISPR were used. Protein expression was assessed via Western Blot 48 hours post-infection. As depicted in FIG. 6, an adenoviral vector encoding HEMO protein was able to infect human dendritic cells in culture to drive expression of the encoded HEMO protein.

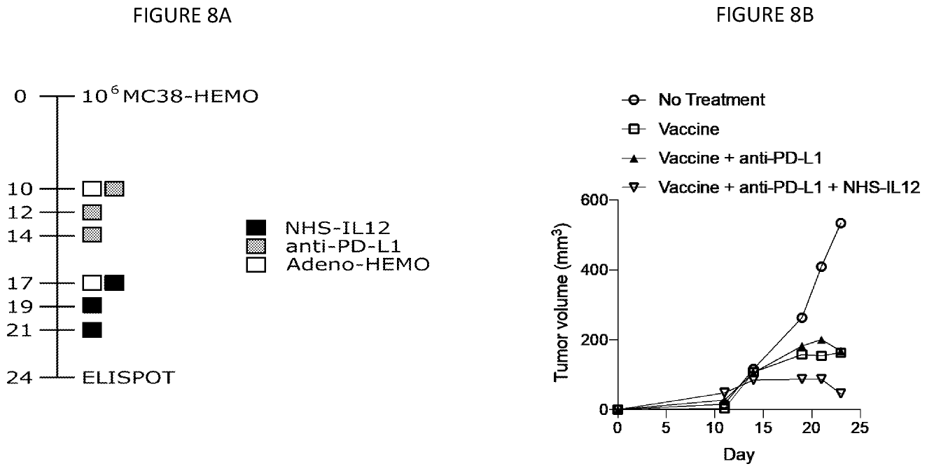

C57BL/c mice were vaccinated with either 1×10¹⁰ control adenovirus or HEMO-encoding adenoviral particles, and boosted 7 days later. Two weeks following the second vaccination, splenocytes were harvested and HEMO-specific immunity was assessed using an IFNγ-ELISPOT assay with 94 individual 15-mer peptides spanning the HEMO protein used as antigen for in vitro stimulation as described above. FIG. 7 shows the results for individual mice in each group, wherein columns correspond to results with each of the 94 individual 15-mer HEMO peptides. These results demonstrate that vaccination of mice with an Adeno-HEMO vector induces immune responses to HEMO peptides in mice.

Murine colon carcinoma MC38 cells were transfected to encode the full-length HEMO protein. Mice bearing MC38-HEMO tumors were left untreated or treated with Adeno-HEMO vaccine, Adeno-HEMO plus anti-PD-L1, or Adeno-HEMO plus anti-PD-L1 and NHS-IL12 (see FIG. 8A). NHS-IL12 is an immunocytokine composed of two IL12 heterodimers fused to the NHS76 antibody (see Strauss et al., Clinical Cancer Research, 25(1): 99-109 (2019)). The average tumor model in each group (n=5 animals per group) is depicted in FIG. 8B. These results demonstrate the anti-tumor effects of vaccination with HEMO peptides, including in combination with other active agents (e.g., NHS-IL12 and/or anti-PD-L1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A peptide comprising the amino acid sequence of any one of SEQ ID NOs: 1-39- and 103, wherein the peptide comprises no more than 21 amino acid residues, and wherein when the peptide comprises SEQ ID NO:1, the peptide comprises at least 15 amino acid residues.

2. The peptide of claim 1, wherein the peptide comprises the amino acid sequence of any one of SEQ ID NOs: 1, 5, 6, 7, 11 or 12.

3. The peptide of claim 1, wherein the peptide consists of any one of SEQ ID NOs: 7-12, 20-28, and 34-39.

4. A nucleic acid encoding the peptide of claim 1.

5. A vector comprising the nucleic acid of claim 4.

6. A vector comprising a nucleic acid that encodes at least two peptides of claim 1.

7. The vector of claim 5, wherein the vector is selected from the group consisting of bacterial, yeast, adenovirus, adeno-associated virus, and poxvirus vectors.

8. An isolated cell comprising (i) one or more of the peptides of claim 1, (ii) one or more nucleic acids encoding (i), or (iii) one or more vectors comprising a nucleic acid of (ii).

9. The cell of claim 8, wherein the cell is human.

10. The cell of claim 8 wherein the cell is an antigen presenting cell or tumor cell.

11. A composition comprising:
(a) one or more of (i) the peptides of claim 1 (ii) nucleic acids encoding (i), (iii) vectors comprising a nucleic acid of (ii) or (iv) cells comprising one or more of (i), (ii), or (iii), and
(b) a pharmaceutically acceptable carrier.

12. The composition of claim 11, further comprising an immunostimulatory/regulatory molecule.

13. The composition of claim 12, wherein the immunostimulatory/regulatory molecule is selected from the group consisting of interleukin (IL)-2, IL-4, IL-6, IL-12, IL-15, IL-15/IL15Ra, IL-15/IL-15Ra-Fc, interferon (IFN)-γ, tumor necrosis factor (TNF)-α, B7.1, B7.2, ICAM-1, LFA-3, CD70, RANTES, G-CSF, OX-40L, 41 BBL, anti-CTLA-4, IDO inhibitor, anti-PDL1, anti-PD1, and combinations thereof.

14. The composition of claim 13, wherein the IL-12 is in the form of an immunocytokine composed of two IL-12 heterodimers fused to the NHS76 antibody.

15. The composition of claim 11, further comprising a chemotherapeutic drug, radioactive agent, antimetabolite, hormone, hormone antagonist, antibiotic, antiviral drug, antifungal drug, or a combination thereof.

16. The composition of claim 11, further comprising an alkylating agent, folate antagonist, purine antagonist, pyrimidine antagonist, spindle poison, topoisomerase inhibitor, apoptosis inducing agent, angiogenesis inhibitor, or a combination thereof.

17. The composition of claim 11, further comprising a vector comprising a nucleic acid encoding tumor associated antigen selected from the group consisting of CEA, MUC1, PSA, and/or Brachyury.

18. The composition of claim 11, further comprising one or more adjuvants.

19. The composition of claim 18, wherein one or more adjuvants is selected from the group consisting of alum, aluminum salts, calcium phosphate, incomplete Freund's adjuvant, QS21, MPL-A, RIBI DETOX™, and combinations thereof.

20. The composition of claim 11, further comprising granulocyte monocyte colony stimulating factor (GM-CSF).

21. The composition of claim 11, further comprising liposomes.

22. A method of inhibiting cancer in a subject comprising administering a therapeutically effective amount of the composition of claim 11 to the subject, wherein the subject is human, and wherein cancer in the subject is inhibited.

23. A method of enhancing an immune response against cancer in a subject comprising administering a therapeutically effective amount of the composition of claim 11 to the subject, wherein the subject is human, and wherein the immune response in the subject is enhanced.

24. A method of inhibiting cancer in a subject comprising:
(a) obtaining lymphocytes from the subject,
(b) stimulating the lymphocytes with the composition of claim 11 to generate cytotoxic T lymphocytes ex vivo, and
(c) administering the cytotoxic T lymphocytes to the subject,
wherein the subject is human, and wherein cancer in the subject is inhibited.

25. A method for inhibiting cancer in a subject comprising:
(a) obtaining dendritic cells from the subject,
(b) treating the dendritic cells with the composition of claim 11 ex vivo, and
(c) administering the treated dendritic cells to the subject,
wherein the subject is human, and wherein cancer in the subject is inhibited.

26. A method for inhibiting cancer in a subject comprising:
(a) obtaining peripheral blood mononuclear cells (PBMCs) from the subject,
(b) isolating dendritic cells from the PBMCs,
(c) treating the dendritic cells with the composition of claim 11 ex vivo,
(d) activating the PBMCs with the treated dendritic cells ex vivo, and
(e) administering the activated PBMCs to the subject,
wherein the subject is human, and wherein cancer in the subject is inhibited.

27. A method for inhibiting cancer in a subject comprising:
(a) obtaining peripheral blood mononuclear cells (PBMCs) from the subject,
(b) isolating dendritic cells from the PBMCs,
(c) treating the dendritic cells with the composition claim 11 ex vivo,
(d) activating the PBMCs with the treated dendritic cells ex vivo,
(e) isolating T lymphocytes from the activated PBMCs ex vivo, and
(f) administering the isolated T lymphocytes to the subject,
wherein the subject is human, and wherein cancer in the subject is inhibited.

28. A method for inhibiting cancer in a subject comprising administering adoptively transferred T cells stimulated in vitro with the composition of claim 11 to the subject, wherein the subject is human, and whereby cancer in the subject is inhibited.

29. A method of inducing an immune response against cancer in a subject comprising:
(a) administering to the subject a first vector comprising a nucleic acid encoding the amino acid sequence of any one of SEQ ID NOs: 1-39 and 103, and
(b) administering to the subject a second vector comprising a nucleic acid encoding the amino acid sequence of any one of SEQ ID NOs: 1-39 and 103,
wherein the subject is human, and wherein an immune response against cancer in the subject is induced.

30. A method for inhibiting cancer in a subject comprising administering T cell receptor (TCR) engineered T cells or TCR engineered NK cells to the subject, wherein the TCR recognizes one or more epitopes of HEMO human endogenous retroviruses (HERVs), wherein the subject is human, and wherein cancer in the subject is inhibited.

31. (canceled)

32. The method of claim 30, wherein the one or more epitopes are selected from the group consisting of SEQ ID NOs: 1-39 and 103.

33. (canceled)

34. (canceled)

35. The composition of claim 11, further comprising podophyllotoxin, nitrosourea, cisplatin, carboplatin, interferon, asparginase, tamoxifen, leuprolide, flutamide, megestrol, mitomycin, bleomycin, doxorubicin, irinotecan, paclitaxel, geldanamycin, cyclophosphamide, or a combination thereof.

36. The composition of claim 18, wherein one or more adjuvants is selected from the group consisting of aluminum phosphate, aluminum hydroxide, aluminum silica, and combinations thereof.


SEQUENCE LISTING (103 sequences)

SEQ ID NO: 1 - protein, 9 aa, Artificial Sequence
  Synthetic
RLLEGNFSL

SEQ ID NO: 2 - protein, 9 aa, Artificial Sequence
  Synthetic
GLGYLVPSL

SEQ ID NO: 3 - protein, 9 aa, Artificial Sequence
  Synthetic
ALLQLTLTA

SEQ ID NO: 4 - protein, 9 aa, Artificial Sequence
  Synthetic
LQLTLTAFL

SEQ ID NO: 5 - protein, 9 aa, Artificial Sequence
  Synthetic
WMYERVWYP

SEQ ID NO: 6 - protein, 9 aa, Artificial Sequence
  Synthetic
WLTGSNLTL

SEQ ID NO: 7 - protein, 9 aa, Artificial Sequence
  Synthetic
RLLEGNFSV

SEQ ID NO: 8 - protein, 9 aa, Artificial Sequence
  Synthetic
GLGYLVPSV

SEQ ID NO: 9 - protein, 9 aa, Artificial Sequence
  Synthetic
ALLQLTLTV

SEQ ID NO: 10 - protein, 9 aa, Artificial Sequence
  Synthetic
LQLTLTAFV

SEQ ID NO: 11 - protein, 9 aa, Artificial Sequence
  Synthetic
WMYERVWYV

SEQ ID NO: 12 - protein, 9 aa, Artificial Sequence
  Synthetic
WLTGSNLTV

SEQ ID NO: 13 - protein, 15 aa, Artificial Sequence
  Synthetic
LSNYALLQLTLTAFL

SEQ ID NO: 14 - protein, 15 aa, Artificial Sequence
  Synthetic
WTYSGQWMYERVWYP

SEQ ID NO: 15 - protein, 15 aa, Artificial Sequence
  Synthetic
WMYERVWYPQAEVQN

SEQ ID NO: 16 - protein, 15 aa, Artificial Sequence
  Synthetic
SFAQVRLLEGNFSLC

SEQ ID NO: 17 - protein, 15 aa, Artificial Sequence
  Synthetic
PTWWLTGSNLTLSVN

SEQ ID NO: 18 - protein, 15 aa, Artificial Sequence
  Synthetic
KWSGRCGLGYLVPSL

SEQ ID NO: 19 - protein, 15 aa, Artificial Sequence
  Synthetic
RCGLGYLVPSLTRYL

SEQ ID NO: 20 - protein, 15 aa, Artificial Sequence
  Synthetic
LSNYALLQLTLTVFL

SEQ ID NO: 21 - protein, 15 aa, Artificial Sequence
  Synthetic
WTYSGQWMYERVWYV

SEQ ID NO: 22 - protein, 15 aa, Artificial Sequence
  Synthetic
WMYERVWYVQAEVQN

SEQ ID NO: 23 - protein, 15 aa, Artificial Sequence
  Synthetic
SFAQVRLLEGNFSVC

SEQ ID NO: 24 - protein, 15 aa, Artificial Sequence
  Synthetic
RTPTWWLTGSNLTVS

SEQ ID NO: 25 - protein, 15 aa, Artificial Sequence
  Synthetic
PTWWLTGSNLTVSVN

SEQ ID NO: 26 - protein, 15 aa, Artificial Sequence
  Synthetic
KWSGRCGLGYLVPSV

SEQ ID NO: 27 - protein, 15 aa, Artificial Sequence
  Synthetic
RCGLGYLVPSVTRYL

SEQ ID NO: 28 - protein, 15 aa, Artificial Sequence
  Synthetic
LSNYALLQLTLTAFV

SEQ ID NO: 29 - protein, 21 aa, Artificial Sequence
  Synthetic
GSLSNYALLQLTLTAFLTILV

SEQ ID NO: 30 - protein, 20 aa, Artificial Sequence
  Synthetic
WTYSGQWMYERVWYPQAEVQ

SEQ ID NO: 31 - protein, 20 aa, Artificial Sequence
  Synthetic
LSFAQVRLLEGNFSLCVENK

SEQ ID NO: 32 - protein, 20 aa, Artificial Sequence
  Synthetic
HRTPTWWLTGSNLTLSVNNS

SEQ ID NO: 33 - protein, 20 aa, Artificial Sequence
  Synthetic
KWSGRCGLGYLVPSLTRYLT

SEQ ID NO: 34 - protein, 21 aa, Artificial Sequence
  Synthetic
GSLSNYALLQLTLTAFVTILV

SEQ ID NO: 35 - protein, 20 aa, Artificial Sequence
  Synthetic
WTYSGQWMYERVWYVQAEVQ

SEQ ID NO: 36 - protein, 20 aa, Artificial Sequence
  Synthetic
LSFAQVRLLEGNFSVCVENK

SEQ ID NO: 37 - protein, 20 aa, Artificial Sequence
  Synthetic
HRTPTWWLTGSNLTVSVNNS

SEQ ID NO: 38 - protein, 20 aa, Artificial Sequence
  Synthetic
KWSGRCGLGYLVPSVTRYLT

SEQ ID NO: 39 - protein, 21 aa, Artificial Sequence
  Synthetic
GSLSNYALLQLTLTVFLTILV

SEQ ID NO: 40 - protein, 9 aa, Artificial Sequence
  Synthetic
FLICSVLSV

SEQ ID NO: 41 - protein, 9 aa, Artificial Sequence
  Synthetic
GIFPLAFFI

SEQ ID NO: 42 - protein, 9 aa, Artificial Sequence
  Synthetic
GLWILVPSA

SEQ ID NO: 43 - protein, 9 aa, Artificial Sequence
  Synthetic
RMKSGTFSV

SEQ ID NO: 44 - protein, 9 aa, Artificial Sequence
  Synthetic
YTLLTIHTV

SEQ ID NO: 45 - protein, 9 aa, Artificial Sequence
  Synthetic
YLSSSQNTI

SEQ ID NO: 46 - protein, 9 aa, Artificial Sequence
  Synthetic
ALSFFLILI

SEQ ID NO: 47 - protein, 9 aa, Artificial Sequence
  Synthetic
AAFLLIWSV

SEQ ID NO: 48 - protein, 9 aa, Artificial Sequence
  Synthetic
SLLDEGIYT

SEQ ID NO: 49 - protein, 9 aa, Artificial Sequence
  Synthetic
LLDEGIYTC

SEQ ID NO: 50 - protein, 9 aa, Artificial Sequence
  Synthetic
AQTALSFFL

SEQ ID NO: 51 - protein, 9 aa, Artificial Sequence
  Synthetic
KVGVFLTPV

SEQ ID NO: 52 - protein, 9 aa, Artificial Sequence
  Synthetic
LLTIHTVHV

SEQ ID NO: 53 - protein, 9 aa, Artificial Sequence
  Synthetic
GIFPLAFFV

SEQ ID NO: 54 - protein, 9 aa, Artificial Sequence
  Synthetic
GLWILVPSV

SEQ ID NO: 55 - protein, 9 aa, Artificial Sequence
  Synthetic
YLSSSQNTV

SEQ ID NO: 56 - protein, 9 aa, Artificial Sequence
  Synthetic
ALSFFLILV

SEQ ID NO: 57 - protein, 9 aa, Artificial Sequence
  Synthetic
SLLDEGIYV

SEQ ID NO: 58 - protein, 9 aa, Artificial Sequence
  Synthetic
LLDEGIYTV

SEQ ID NO: 59 - protein, 9 aa, Artificial Sequence
  Synthetic
AQTALSFFV

SEQ ID NO: 60 - protein, 14 aa, Artificial Sequence
  Synthetic
MKAQTALSFFLILI

SEQ ID NO: 61 - protein, 15 aa, Artificial Sequence
  Synthetic
QTALSFFLILITSLS

SEQ ID NO: 62 - protein, 15 aa, Artificial Sequence
  Synthetic
SLSGSQGIFPLAFFI

SEQ ID NO: 63 - protein, 15 aa, Artificial Sequence
  Synthetic
SQGIFPLAFFIYVPM

SEQ ID NO: 64 - protein, 15 aa, Artificial Sequence
  Synthetic
LFFRRVSLLDEGIYT

SEQ ID NO: 65 - protein, 15 aa, Artificial Sequence
  Synthetic
RRVSLLDEGIYTCYV

SEQ ID NO: 66 - protein, 15 aa, Artificial Sequence
  Synthetic
NKVVLKVGVFLTPVM

SEQ ID NO: 67 - protein, 15 aa, Artificial Sequence
  Synthetic
VLKVGVFLTPVMKYE

SEQ ID NO: 68 - protein, 15 aa, Artificial Sequence
  Synthetic
KRNTNSFLICSVLSV

SEQ ID NO: 69 - protein, 15 aa, Artificial Sequence
  Synthetic
NSFLICSVLSVYPRP

SEQ ID NO: 70 - protein, 15 aa, Artificial Sequence
  Synthetic
WSRMKSGTFSVLAYY

SEQ ID NO: 71 - protein, 15 aa, Artificial Sequence
  Synthetic
LAYYLSSSQNTIINE

SEQ ID NO: 72 - protein, 15 aa, Artificial Sequence
  Synthetic
SDEYTLLTIHTVHVE

SEQ ID NO: 73 - protein, 15 aa, Artificial Sequence
  Synthetic
TASHNKGLWILVPSA

SEQ ID NO: 74 - protein, 15 aa, Artificial Sequence
  Synthetic
GLWILVPSAILAAFL

SEQ ID NO: 75 - protein, 15 aa, Artificial Sequence
  Synthetic
VPSAILAAFLLIWSV

SEQ ID NO: 76 - protein, 15 aa, Artificial Sequence
  Synthetic
SAILAAFLLIWSVKC

SEQ ID NO: 77 - protein, 14 aa, Artificial Sequence
  Synthetic
MKAQTALSFFLILV

SEQ ID NO: 78 - protein, 15 aa, Artificial Sequence
  Synthetic
QTALSFFLIVITSLS

SEQ ID NO: 79 - protein, 15 aa, Artificial Sequence
  Synthetic
SLSGSQGIFPLAFFV

SEQ ID NO: 80 - protein, 15 aa, Artificial Sequence
  Synthetic
SQGIFPLAFFVYVPM

SEQ ID NO: 81 - protein, 15 aa, Artificial Sequence
  Synthetic
LFFRRVSLLDEGIYV

SEQ ID NO: 82 - protein, 15 aa, Artificial Sequence
  Synthetic
RRVSLLDEGIYVCYV

SEQ ID NO: 83 - protein, 15 aa, Artificial Sequence
  Synthetic
LAYYLSSSQNTVINE

SEQ ID NO: 84 - protein, 15 aa, Artificial Sequence
  Synthetic
TASHNKGLWILVPSV

SEQ ID NO: 85 - protein, 15 aa, Artificial Sequence
  Synthetic
GLWILVPSVILAAFL

SEQ ID NO: 86 - protein, 20 aa, Artificial Sequence
  Synthetic
MKAQTALSFFLILITSLSGS

SEQ ID NO: 87 - protein, 20 aa, Artificial Sequence
  Synthetic
SLSGSQGIFPLAFFIYVPMN

SEQ ID NO: 88 - protein, 21 aa, Artificial Sequence
  Synthetic
LFFRRVSLLDEGIYTCYVGTA

SEQ ID NO: 89 - protein, 20 aa, Artificial Sequence
  Synthetic
TNKVVLKVGVFLTPVMKYEK

SEQ ID NO: 90 - protein, 20 aa, Artificial Sequence
  Synthetic
KRNTNSFLICSVLSVYPRPI

SEQ ID NO: 91 - protein, 20 aa, Artificial Sequence
  Synthetic
FKVTWSRMKSGTFSVLAYYL

SEQ ID NO: 92 - protein, 20 aa, Artificial Sequence
  Synthetic
FSVLAYYLSSSQNTIINESR

SEQ ID NO: 93 - protein, 22 aa, Artificial Sequence
  Synthetic
NISSDEYTLLTIHTVHVEPSQE

SEQ ID NO: 94 - protein, 20 aa, Artificial Sequence
  Synthetic
TASHNKGLWILVPSAILAAF

SEQ ID NO: 95 - protein, 20 aa, Artificial Sequence
  Synthetic
VPSAILAAFLLIWSVKCCRA

SEQ ID NO: 96 - protein, 20 aa, Artificial Sequence
  Synthetic
MKAQTALSFFLILVTSLSGS

SEQ ID NO: 97 - protein, 20 aa, Artificial Sequence
  Synthetic
SLSGSQGIFPLAFFVYVPMN

SEQ ID NO: 98 - protein, 21 aa, Artificial Sequence
  Synthetic
LFFRRVSLLDEGIYVCYVGTA

SEQ ID NO: 99 - protein, 20 aa, Artificial Sequence
  Synthetic
FSVLAYYLSSSQNTVINESR

SEQ ID NO: 100 - protein, 20 aa, Artificial Sequence
  Synthetic
TASHNKGLWILVPSVILAAF

SEQ ID NO: 101 - protein, 9 aa, Artificial Sequence
  Synthetic
WLLPGTSTV

SEQ ID NO: 102 - protein, 9 aa, Artificial Sequence
  Synthetic
SYLIRALTL

SEQ ID NO: 103 - protein, 15 aa, Artificial Sequence
  Synthetic
RTPTWWLTGSNLTLS